(19) United States
(12) Patent Application Publication
(10) Pub. No.: US 2008/0188460 A1
(43) Pub. Date: Aug. 7, 2008

(54) Tricyclic compounds, a process for their preparation and pharmaceutical compositions containing them

(75) Inventors: Patrick Casara, Vilennes Sur Seine (FR); Thierry Le Diguarher, Rueil Malmaison (FR); Olivier Geneste, Rueil Malmaison (FR); John Hickman, Paris (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Courbevoie Cedex (FR)

(21) Appl. No.: 12/011,225

(22) Filed: Jan. 24, 2008

(30) Foreign Application Priority Data
Feb. 2, 2007 (FR) 07/00741

(51) Int. Cl. A61K 31/55; C07D 241/36; A61K 31/4985; C07D 285/20; A61K 31/5377; A61P 35/00; A61K 31/541; C07D 295/00; A61K 31/5415; C07D 223/02

(52) U.S. Cl. 514/215; 544/344; 514/250; 544/12; 514/223.2; 540/578; 544/115; 514/233.2; 544/60; 514/228.2

(57) ABSTRACT

Compounds of formula (I):

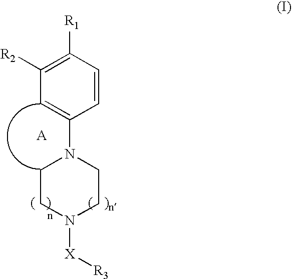

wherein
A represents a 5, 6 or 7-membered (hetero)aromatic or non-aromatic ring,
n and n′ represent 0, 1 or 2
X represents an alkylene chain as defined in the description,
R₃ represents an aryl or heteroaryl group,
one of the groups R₁ and R₂ represents a hydrogen atom and the other represents a group of formula (II) as defined in the description.

Medicinal products containing the same which are useful in treating conditions involving a defect in apoptosis.

The present invention relates to new tricyclic compounds, to a process for their preparation and to pharmaceutical compositions containing them.

The compounds of the present invention are new and have very valuable pharmacological characteristics in the field of apoptosis and cancerology.

Apoptosis, or programmed cell death, is a crucial physiological process in embryo development and in maintaining tissue homeostasis.

Apoptotic-type cell death causes morphological changes, such as condensation of the nucleus, DNA fragmentation and biochemical phenomena, such as the activation of caspases which cause damage to key structural components of the cell, so inducing its disassembly and death. Regulation of the process of apoptosis is complex and involves the activation or repression of several intracellular signalling pathways (Cory S. et al., Nature Review Cancer, 2002, 2, 647-656).

Disturbances in apoptosis are involved in certain pathologies. Increased apoptosis is associated with neurodegenerative diseases, such as Parkinson's disease, Alzheimer's disease and ischaemia. Conversely, deficiencies in the execution of apoptosis play a significant role in the development of cancers and their chemoresistance, in auto-immune diseases, inflammatory diseases and viral infections. Accordingly, absence of apoptosis is one of the phenotypic signatures of cancer (Hanahan D. et al., Cell 2000, 100, 57-70).

The compounds of the present invention, in addition to being new, have pro-apoptotic properties that mean they can be used in pathologies involving a defect in apoptosis, such as, for example, in the treatment of cancer.

The present invention relates more especially to a compound of formula (I):

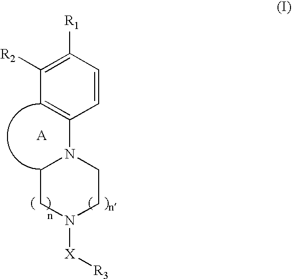

wherein:
A represents a 5, 6 or 7-membered aromatic or non-aromatic ring which may contain 1 or 2 hetero atoms selected from oxygen, sulphur and nitrogen, it being possible for the latter to be substituted by a linear or branched (C₁-C₆)alkyl group, it being understood that the ring A so defined cannot contain 2 sulphur atoms or 2 oxygen atoms and that one of the ring members may be a C═O group,
n and n′, which may be identical or different, represent 0, 1 or 2, where 0<n+n′<4,
R₃ represents an aryl or heteroaryl group,
X represents a linear or branched alkylene chain containing from 1 to 6 carbon atoms, one or two of which carbon atoms may be replaced by an oxygen atom, a cycloalkylene group, an arylene group, a heteroarylene group or SO₂ group,
one of the groups R₁ and R₂ represents a hydrogen atom and the other represents a group of formula (II):

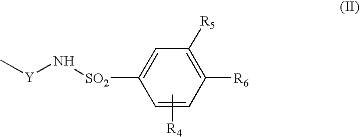

wherein
Y represents a C═O or CH₂ group,
R₅ represents a hydrogen atom in which case R₆ represents a hydrogen atom or a —NR₇R′₇ or —CH₂—NR₇R′₇ group wherein each of R₇ and R′₇, which may be identical or different, independently of the other represents a hydrogen atom or a linear or branched (C₁-C₆)alkyl group substituted by one or more aryl, heteroaryl, aryloxy, heteroaryloxy, arylthio, heteroarylthio, heterocycloalkyl or —NR₁₀R′₁₀ groups wherein:
   R₁₀ and R′₁₀, which may be identical or different, are selected from hydrogen, linear or branched (C₁-C₆)alkyl, linear or branched (C₁-C₆)alkoxy, aryl and heteroaryl, or
   R₁₀ and R′₁₀ form a saturated or unsaturated cyclic or bicyclic group which may be substituted by a hetero atom selected from oxygen, nitrogen and sulphur, it being understood that one or more of the ring members may represent a C═O group or may be substituted as indicated in the definition of a heterocycloalkyl given below,
or R₅ and R₆ form with the two carbon atoms carrying them an aromatic or non-aromatic ring containing 5 or 6 ring members, one nitrogen atom of which being in the position para to the SO₂ group, and which may contain in addition to the nitrogen atom a further nitrogen atom and/or a SO₂ group, the ring so defined being substituted by an R₇ group as defined above,
R₄ represents a halogen atom or an NO₂, R₈, SO₂—R₉, linear or branched (C₁-C₆)-alkyl or linear or branched (C₁-C₆)alkoxy group, wherein R₈ may have any of the values of R₇ as defined above,
R₉ represents an amino group or a linear or branched (C₁-C₆)alkyl group optionally substituted by one or more halogen atoms,

it being understood that:
“aryl” is understood to mean a phenyl, naphthyl or biphenyl group,
“heteroaryl” is understood to mean any mono- or bi-cyclic group having at least one aromatic moiety and containing from 5 to 10 ring members and which may contain from 1 to 3 hetero atoms selected from oxygen, sulphur and nitrogen, such as the groups furan, thiophene, pyrrole, imidazoline, pyridine, quinoline, isoquinoline, chroman, indole, benzothiophene, benzofuran, 1,3-benzodioxole and 2,3-dihydro-1,4-benzodioxine,
“heterocycloalkyl” is understood to mean any mono- or bi-cyclic non-aromatic group containing from 4 to 10 ring members and which may contain from 1 to 3 hetero atoms selected from oxygen, sulphur and nitrogen,
“cycloalkyl” is understood to mean any mono- or bi-cyclic non-aromatic group containing from 4 to 10 ring members,
   it being possible for the aryl, heteroaryl, heterocycloalkyl and cycloalkyl groups so defined to be substituted by from 1 to 3 groups selected from linear or branched (C₁-C₆)alkyl optionally substituted by a hydroxy or amino group, linear or branched (C₁-C₆)alkoxy, hydroxy, carboxy, formyl, nitro, cyano, amino, linear or branched polyhalo-(C₁-C₆)alkyl, alkoxycarbonyl and halogen atoms,
“arylene”, “heteroarylene” and “cycloalkylene” are understood to mean, respectively, an aryl, heteroaryl or cycloalkyl group as defined above, inserted instead of a carbon atom of the alkylene chain,
   its enantiomers and diastereoisomers, and addition salts thereof with a pharmaceutically acceptable acid or base.

Among the pharmaceutically acceptable acids there may be mentioned by way of non-limiting example hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, etc . . .

Among the pharmaceutically acceptable bases there may be mentioned by way of non-limiting example sodium hydroxide, potassium hydroxide, triethylamine, tert-butylamine, etc.

Y advantageously represents a C═O group.

The preferred value for n and n′ is 1.

The preferred R₄ groups are the groups NO₂ and SO₂CF₃.

The preferred X—R₃ groups are the ([1,1′-biphenyl]-2-yl)methyl groups optionally substituted by one or more groups selected from halogen, cyano, amino, aminomethyl and trifluoromethyl.

R₅ preferably represents a hydrogen atom.

The preferred R₇ groups are the groups 1-(N,N-dimethylamino)-4-(phenylsulphanyl)-butan-3-yl and 1-(NR₁₀R′₁₀)-4-(phenylsulphanyl)-butan-3-yl, R₁₀ and R′₁₀ being such that they form a saturated or unsaturated cyclic or bicyclic group optionally substituted by a hetero atom selected from oxygen, nitrogen and sulphur.

R′₇ advantageously represents a hydrogen atom.

The preferred

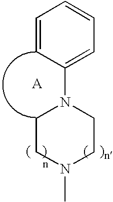

group is the group

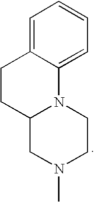

More especially, the invention relates to compounds of formula (I) which are:

N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide,
N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide bistrifluoroacetate,
N-({(4aS,R)-3-[(4′-chloro-[1,1′-biphenyl]-3-yl)methyl]-2,3,4,4a,5,6-hexahydro-1H-pyrazino[1,2-a]quinolin-8-yl}carbonyl)-3-nitro-4-{[2-(phenylsulphanyl)ethyl]-amino}benzenesulphonamide,
N-({(4aS,R)-3-[(4′-chloro-[1,1′-biphenyl]-3-yl)methyl]-2,3,4,4a,5,6-hexahydro-1H-pyrazino[1,2-a]quinolin-8-yl}carbonyl)-3-nitro-4-{[2-(phenylsulphanyl)ethyl]-amino}benzenesulphonamide hydrochloride,
N-({(4aS,R)-3-[(4′-chloro-[1,1′-biphenyl]-4-yl)methyl]-2,3,4,4a,5,6-hexahydro-1H-pyrazino[1,2-a]quinolin-8-yl}carbonyl)-3-nitro-4-{[2-(phenylsulphanyl)ethyl]-amino}benzenesulphonamide,
N-({(4aS,R)-3-[(4′-chloro-[1,1′-biphenyl]-4-yl)methyl]-2,3,4,4a,5,6-hexahydro-1H-pyrazino[1,2-a]quinolin-8-yl}carbonyl)-3-nitro-4-{[2-(phenylsulphanyl)-ethyl]amino}benzenesulphonamide hydrochloride,
N-{[(4aS,R)-3-([1,1′-biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenyl-sulphanyl)methyl]propyl}amino)-3-nitrobenzenesulphonamide,
N-{[(4aS,R)-3-([1,1′-biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenyl-sulphanyl)methyl]propyl}amino)-3-nitrobenzenesulphonamide bis(hydrochloride),
N-{[(4aS,R)-3-(2-benzylbenzyl)-2,3,4,4a,5,6-hexahydro-1H-pyrazino[1,2-a]-quinolin-8-yl]carbonyl}-3-nitro-4-{[2-(phenylsulphanyl)ethyl]amino}benzene-sulphonamide,
N-{[(4aS,R)-3-(2-benzylbenzyl)-2,3,4,4a,5,6-hexahydro-1H-pyrazino[1,2-a]-quinolin-8-yl]carbonyl}-3-nitro-4-{[2-(phenylsulphanyl)ethyl]amino}benzene-sulphonamide hydrochloride,
3-nitro-N-({(4aS,R)-3-[2-(2-phenylethyl)benzyl]-2,3,4,4a,5,6-hexahydro-1H-pyrazino[1,2-a]quinolin-8-yl}carbonyl)-4-{[2-(phenylsulphanyl)ethyl]amino}-benzenesulphonamide,
N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)-3-nitro-4-{[2-(phenylsulphanyl)ethyl]-amino}benzenesulphonamide,
N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)-3-nitro-4-[(2-phenoxyethyl)amino]-benzenesulphonamide,
N-{[(4aS,R)-3-([1,1′-biphenyl]-2-ylmethyl)-2,3,4,4a,5,6-hexahydro-1H-pyrazino-[1,2-a]quinolin-8-yl]carbonyl}-3-nitro-4-[(3-phenylpropyl)amino]benzene-sulphonamide,
4-[(2-anilinoethyl)amino]-N-{[(4aS,R)-3-([1,1′-biphenyl]-2-ylmethyl)-2,3,4,4a,5,6-hexahydro-1H-pyrazino[1,2-a]quinolin-8-yl]carbonyl}-3-nitrobenzene-sulphonamide,
N-{[(4aS,R)-3-([1,1′-biphenyl]-2-ylmethyl)-2,3,4,4a,5,6-hexahydro-1H-pyrazino-[1,2-a]quinolin-8-yl]carbonyl}-4-{[3-(dimethylamino)propyl][2-(phenylsulphanyl)-ethyl]amino}-3-nitrobenzenesulphonamide,
N-{[(4aS,R)-3-([1,1′-biphenyl]-2-ylmethyl)-2,3,4,4a,5,6-hexahydro-1H-pyrazino-[1,2-a]quinolin-8-yl]carbonyl}-4-{[3-(dimethylamino)propyl][2-(phenylsulphanyl)-ethyl]amino}-3-nitrobenzenesulphonamide hydrochloride,
N-({(4aS, R)-3-[(4′-chloro-[1,1′-biphenyl]-2-yl)methyl]-2,3,4,4a,5,6-hexahydro-1H-pyrazino[1,2-a]quinolin-8-yl}carbonyl)-4-{[3-(dimethylamino)propyl]amino}-3-nitrobenzenesulphonamide,
N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)benzenesulphonamide,
4-{[(2-aminoethyl)(2-phenylethyl)amino]methyl}-N-({(4aS,R)-3-[(4′-chloro-[1,1′-biphenyl]-2-yl)methyl]-2,3,4,4a,5,6-hexahydro-1H-pyrazino[1,2-a]quinolin-8-yl}-carbonyl)benzenesulphonamide,
4-{[(2-aminoethyl)(2-phenylethyl)amino]methyl}-N-({(4aS,R)-3-[(4′-chloro-[1,1′-biphenyl]-2-yl)methyl]-2,3,4,4a,5,6-hexahydro-1H-pyrazino[1,2-a]quinolin-8-yl}-carbonyl)benzenesulphonamide tris(trifluoroacetate),
N-({(4aS,R)-3-[(4′-chloro-[1,1′-biphenyl]-2-yl)methyl]-2,3,4,4a,5,6-hexahydro-1H-pyrazino[1,2-a]quinolin-7-yl}carbonyl)-4-({(1R)-3-(dimethylamino)-1-[(phenyl-sulphanyl)methyl]propyl}amino)-3-nitrobenzenesulphonamide,
N-{[(4aS,R)-3-([1,1′-biphenyl]-2-ylmethyl)-2,3,4,4a,5,6-hexahydro-1H-pyrazino-[1,2-a]quinolin-7-yl]carbonyl}-4-({(1R)-3-(dimethylamino)-1-[(phenylsulphanyl)-methyl]propyl}amino)-3-nitrobenzenesulphonamide,
N-{[2-([1,1′-biphenyl]-2-ylmethyl)-1,2,3,4-tetrahydropyrazino[1,2-a]indol-8-yl]-carbonyl}-4-({(1R)-3-(dimethylamino)-1-[(phenylsulphanyl)methyl]propyl}-amino)-3-nitrobenzenesulphonamide,
N-{[2-([1,1′-biphenyl]-2-ylmethyl)-1,2,3,4-tetrahydropyrazino[1,2-a]indol-8-yl]-carbonyl}-4-({(1R)-3-(dimethylamino)-1-[(phenylsulphanyl)methyl]propyl}-amino)-3-nitrobenzenesulphonamide bis(hydrochloride),
N-({2-[(4′-chloro-[1,1′-biphenyl]-2-yl)methyl]-1,2,3,4-tetrahydropyrazino[1,2-a]-indol-8-yl}carbonyl)-4-({(1R)-3-(dimethylamino)-1-[(phenylsulphanyl)methyl]-propyl}amino)-3-nitrobenzenesulphonamide,
N-({2-[(4′-chloro-[1,1′-biphenyl]-2-yl)methyl]-1,2,3,4-tetrahydropyrazino [1,2-a]-indol-8-yl}carbonyl)-4-({(1R)-3-(dimethylamino)-1-[(phenylsulphanyl)methyl]-propyl}amino)-3-nitrobenzenesulphonamide bis(hydrochloride),
N-({(10aS,R)-2-[(4′-chloro-[1,1′-biphenyl]-2-yl)methyl]-1,2,3,4,10,10a-hexahydro-pyrazino[1,2-a]indol-8-yl}carbonyl)-4-({(1R)-3-(dimethylamino)-1-[(phenyl-sulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide bis(hydrochloride),
N-({(4aS)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide,
N-({(4aS)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide bis(hydrochloride),
N-{[(4aS)-3-([1,1′-biphenyl]-2-ylmethyl)-2,3 ,4,4a,5,6-hexahydro-1H-pyrazino[1,2-a]quinolin-8-yl]carbonyl}-4-({(1R)-3-(dimethylamino)-1-[(phenylsulphanyl)-methyl]propyl}amino)-3-nitrobenzenesulphonamide,
N-{[(4aS)-3-([1,1′-biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)-methyl]propyl}amino)-3-nitrobenzenesulphonamide bis(hydrochloride),
N-{[(4aR)-3-([1,1′-biphenyl]-2-ylmethyl)-2,3,4,4a,5,6-hexahydro-1H-pyrazino-[1,2-a]quinolin-8-yl]carbonyl}-4-({(1R)-3-(dimethylamino)-1-[(phenylsulphanyl)-methyl]propyl}amino)-3-nitrobenzenesulphonamide,
N-{[(4aR)-3-([1,1′-biphenyl]-2-ylmethyl)-2,3,4,4a,5,6-hexahydro-1H-pyrazino-[1,2-a]quinolin-8-yl]carbonyl}-4-({(1R)-3-(dimethylamino)-1-[(phenylsulphanyl)-methyl]propyl}amino)-3-nitrobenzenesulphonamide tris(hydrochloride),
N-{[(4aS,R)-3-([1,1′-biphenyl]-2-ylsulphonyl)-2,3,4,4a,5,6-hexahydro-1H-pyrazino[1,2-a]quinolin-8-yl]carbonyl}-3-nitro-4-{[2-(phenylsulphanyl)ethyl]-amino}benzenesulphonamide,
N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)-4-{(1R)-3-(dimethylamino)-1-[(phenyl-sulphanyl)methyl]propyl}-3,4-dihydro-2H-1,2,4-benzothiadiazine-7-sulphonamide 1,1-dioxide,
N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)-4-{(1R)-3-(dimethylamino)-1-[(phenyl-sulphanyl)methyl]propyl}-4H-1,2,4-benzothiadiazine-7-sulphonamide 1,1-dioxide,
N-({(10aα)-2-[(4′-chloro-[1,1′-biphenyl]-2-yl)methyl]-1,2,3,4,10,10a-hexa-hydropyrazino[1,2-a]indol-8-yl}carbonyl)-4-({(1R)-3-(dimethylamino)-1-[(phenylsulphanyl)methyl]propyl}amino)-3-nitrobenzenesulphonamide bishydrochloride,
N-({(10aβ)-2-[(4′-chloro-[1,1′-biphenyl]-2-yl)methyl]-1,2,3,4,10,10a-hexahydropyrazino[1,2-a]indol-8-yl}carbonyl)-4-({(1R)-3-(dimethylamino)-1-[(phenylsulphanyl)methyl]propyl}amino)-3-nitrobenzenesulphonamide bishydrochloride,
N-({(10aα)-2-[(4′-chloro-[1,1′-biphenyl]-2-yl)methyl]-1,2,3,4,10,10a-hexahydropyrazino[1,2-a]indol-8-yl}carbonyl)-4-({(1R)-3-(4-morpholinyl)-1-[(phenylsulphanyl)methyl]propyl}amino)-3-nitrobenzenesulphonamide bishydrochloride,
N-({(10aα)-2-[(4′-chloro-[1,1′-biphenyl]-2-yl)methyl]-1,2,3,4,10,10a-hexahydropyrazino[1,2-a]indol-8-yl}carbonyl)-4-({(1R)-3-(4-morpholinyl)-1-[(phenylsulphanyl)methyl]propyl}amino)-3-[(trifluoromethyl)sulphonyl]benzene-sulphonamide bishydrochloride,
N-[((4aR)-3-{[2-(4-chlorophenyl)-5,5-dimethyl-1-cyclohexen-1-yl]methyl}-2,3,4,4a,5,6-hexahydro-1H-pyrazino[1,2-a]quinolin-8-yl)carbonyl]-4-({(1R)-3-(4-morpholinyl)-1-[(phenylsulphanyl)methyl]propyl}amino)-3-[(trifluoromethyl)-sulphonyl]benzenesulphonamide bishydrochloride,
N-[((10aβ)-2-{[2-(4-chlorophenyl)-5,5-dimethyl-1-cyclohexen-1-yl]methyl}-1,2,3,4,10,10a-hexahydropyrazino [1,2-a] indol-8-yl)carbonyl]-4-({(1R)-3-(4-morpholinyl)-1-[(phenylsulphanyl)methyl]propyl}amino)-3-[(trifluoromethyl)-sulphonyl]benzenesulphonamide bishydrochloride,
N-[((4aR)-3-{[4-(4-chlorophenyl)-3-pyridyl]methyl}-2,3,4,4a,5,6-hexahydro-1H-pyrazino[1,2-a]quinolin-8-yl)carbonyl]-4-({(1R)-2-(dimethylamino)-1-[(phenylsulphanyl)methyl]ethyl}amino)-3-nitrobenzenesulphonamide trihydrochloride,
N-({(4aR)-3-[(4-amino-4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitro-benzenesulphonamide trihydrochloride,
N-[((4aR)-3-{[4-(aminomethyl)-4′-chloro-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzene-sulphonamide trihydrochloride,
N-[((4aR)-3-{[3′-fluoro-4′-chloro-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzene-sulphonamide bishydrochloride,
N-[((4aR)-3-{[4′-(trifluoromethyl)-[1,1′-biphenyl]-2-yl]methyl}-2,3,4,4a,5,6-hexahydro-1H-pyrazino [1,2-a]quinolin-8-yl)carbonyl]-4-({(1R)-3-(dimethylamino)-1-[(phenylsulphanyl)methyl]propyl}amino)-3-nitro-benzenesulphonamide bishydrochloride,
N-[((4aR)-3-{[4′-cyano-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide bishydrochloride,
N-({(4aR)-3-[2-(1,3-benzodioxol-5-yl)benzyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-[(trifluoromethyl)sulphonyl]-benzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-methyl-1-piperazinyl)-1H-[(phenylsulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-piperidyl)-1-[(phenylsulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-pyrrolidinyl)-1-[(phenylsulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(3,6-dihydro-1(2H)-pyridyl-1-[(phenylsulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-azepanyl)-1-[(phenyl-sulphanyl)methyl]propyl}amino)-3-nitrobenzenesulphonamide bishydrochloride,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3--((1R,5S)-3-azabicyclo-[3.1.0]hex-3-yl)-1-[(phenylsulphanyl)methyl]propyl}amino)-3-nitrobenzene-sulphonamide bishydrochloride,
sodium N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide.

The enantiomers, diastereoisomers and addition salts thereof with a pharmaceutically acceptable acid or base of the preferred compounds of the invention form an integral part of the invention.

The invention relates also to a process for the preparation of a compound of formula (I), characterised in that there is used as starting material a compound of formula (III):

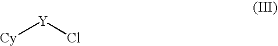

wherein Y is as defined for formula (I) and Cy represents a fused tricyclic system of formula (IV):

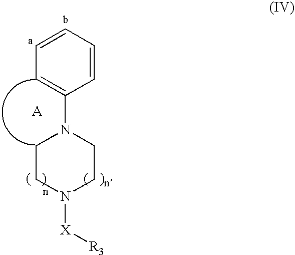

wherein A, X, n, n′ and R₃ are as defined for formula (I), the —Y—Cl group being attached in the a or b position of the tricyclic system so defined,

which compound of formula (III) is condensed, in a basic medium in the presence or absence of a coupling agent, with a compound of formula (V):

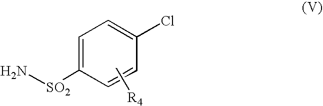

wherein R₄ is as defined for formula (I),

to obtain a compound of formula (VI):

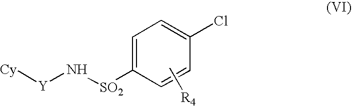

wherein Cy, Y and R₄ are as defined hereinbefore,

which is condensed with a compound of formula HNR₇R′₇ wherein R₇ and R′₇ are as defined for formula (I) to yield a compound of formula (I/a), a particular case of the compound of formula (I):

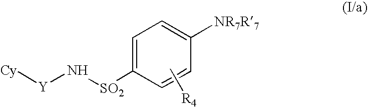

wherein Cy, Y, R₄, R₇ and R′₇ are as defined hereinbefore,

which may be purified according to a conventional separation technique, which is converted, if desired, into addition salts thereof with a pharmaceutically acceptable acid or base and which is optionally separated into its isomers according to a conventional separation technique.

The compounds of formulae (III) and (V) are either commercial compounds or are accessible to a person skilled in the art by conventional chemical reactions described in the literature.

An advantageous variant relates to a process for the preparation of a compound of formula (I), characterised in that there is used as starting material a compound of formula (III′):

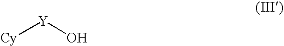

wherein Y is as defined for formula (I) and Cy represents a fused tricyclic system of formula (IV):

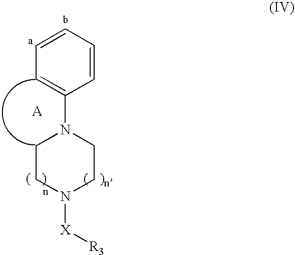

wherein A, X, R₃, n and n′ are as defined for formula (I), the —Y—OH group being attached in the a or b position of the tricyclic system so defined,

which compound of formula (III′) is condensed, in a basic medium in the presence of a coupling agent, with a compound of formula (VII):

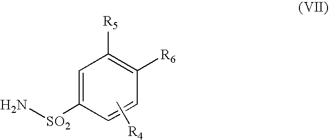

wherein R₄, R₅ and R₆ are as defined for formula (I),

to yield a compound of formula (I) which may be purified according to a conventional separation technique, which is converted, if desired, into addition salts thereof with a pharmaceutically acceptable acid or base and which is optionally separated into its isomers according to a conventional separation technique.

The compounds of formulae (III′) and (VII) are either commercial or are accessible to a person skilled in the art by conventional chemical reactions described in the literature.

The pharmacological study of the compounds of the invention has shown that they have pro-apoptotic properties. The ability to reactivate the apoptotic process in cancerous cells is of major therapeutic interest in the treatment of cancers.

More especially, the compounds according to the invention will be useful in the treatment of chemo- or radio-resistant cancers, and in malignant haemopathies and in small-cell lung cancer.

Among the treatment of cancers envisaged there may be mentioned, without imposing any limitation, cancers of the bladder, brain, breast and uterus, chronic lymphoid leukaemias, cancers of the colon, cesophagus and liver, lymphoblastic leukaemias, follicular lymphomas, melanomas, malignant haemopathies, myelomas, ovarian cancers, non-small-cell lung cancers, prostate cancers and small-cell lung cancers.

The present invention relates also to pharmaceutical compositions comprising at least one compound of formula (I) on its own or in combination with one or more pharmaceutically acceptable excipients.

Among the pharmaceutical compositions according to the invention there may be mentioned more especially those that are suitable for oral, parenteral, nasal, per- or transcutaneous, rectal, perlingual, ocular or respiratory administration, especially tablets or dragees, sublingual tablets, sachets, packets, gelatin capsules, glossettes, lozenges, suppositories, creams, ointments, dermal gels, and drinkable or injectable ampoules.

The dosage varies according to the sex, age and weight of the patient, the route of administration, the nature of the therapeutic indication, or any associated treatments, and ranges from 0.01 mg to 1 g per 24 hours in one or more administrations.

Moreover, the present invention relates also to the combination of a compound of formula (I) with an anticancer agent selected from genotoxic agents, mitotic poisons, anti-metabolites, proteasome inhibitors and kinase inhibitors, and to the use of that type of combination in the manufacture of medicaments for use in the treatment of cancer.

The compounds of the invention may also be used in combination with radiotherapy in the treatment of cancer.

The following Preparations and Examples illustrate the invention but do not limit it in any way.

### Preparation 1: (4aS,R)-3-[(4′-Chloro-1,1′-biphenyl]-2-yl)methyl]-2,3,4,4a,5,6-hexahydro-1H-pyrazino[1,2-a]quinoline8-carboxylic acid hydrochloride

### Step A: 6-Methoxy-2-quinolinecarbaldehyde

Selenium oxide is added in portions to a solution of 6-methoxy-2-methylquinoline (42 g) in 400 ml of a mixture of dioxane/H₂O (5%) and then the whole is heated at reflux overnight. The mixture is left to cool, the metal is removed by filtration and concentration to dryness is carried out. The resulting dark brown solid is purified by chromatography over a silica column (heptane/AcOEt 80/20) to yield the title product in the form of a white solid.

### Step B: 2-{[(6-Methoxy-2-quinolyl)methyl]amino}ethanol

9.5 ml of 3-aminopropanol are added to a suspension of the compound obtained in Step A (28 g) in 200 ml of EtOH, and the whole is refluxed using a Dean-Stark apparatus overnight. The reaction mixture is then concentrated to dryness and taken up in a volume of 200 ml of EtOH at 0° C,; 14 g of NaBH₄ are then added in portions. The whole is then heated at reflux overnight. The reaction mixture is then concentrated to dryness, hydrolysed with H₂O and extracted with CH₂Cl₂. Drying over MgSO₄ and concentration to dryness yield an oil which gradually crystallises. The crystals are then triturated in diisopropyl ether, filtered and dried to yield the title product in the form of beige crystals.

### Step C: 2-{[(6-Methoxy-1,2,3,4-tetrahydro-2-quinolyl)methyl]amino}ethanol

169 g of Raney nickel are added in portions to a solution of the compound obtained in Step B (33 g) in a mixture of 50/50 MeOH/KOH 1M (1.21). The whole is then stirred at ambient temperature overnight. The metal is then removed by filtration and the filtrate is concentrated to dryness. The residue is then taken up in CH₂Cl₂, hydrolysed with H₂O and then extracted several times with CH₂Cl₂. The extracts are then combined, dried over MgSO₄, filtered and concentrated to dryness. The resulting crystals are triturated in diisopropyl ether, filtered and dried to yield the title compound in the form of white crystals.

### Step D: (4aS,R)-8-Methoxy-2,3,4,4a,5,6-hexahydro-1H-pyrazino[1,2-a]quinoline

P₂O₅ (18 g) is added to a suspension of the compound obtained in Step C (10 g) in 200 ml of o-xylene. The whole is then heated at 150° C. overnight. The mixture is left to cool, concentration to dryness is carried out and then cold hydrolysis is carried out slowly with H₂O, 5N NaOH is then added slowly without heating and the whole is stirred at ambient temperature for 30 minutes. The reaction mixture is then extracted several times with CH₂Cl₂, dried over MgSO₄ and concentrated to dryness to obtain a brown oil corresponding to the title product, which is used directly without purification in the following Step.

### Step E: (4aS,R)-3-Benzyl-8-methoxy-2,3,4,4a,5,6-hexahydro-1H-pyrazino[1,2-a]-quinoline

The compound obtained in Step D (7 g) is dissolved in 100 ml of DMF, and there are then added in succession 8.85 g of K₂CO₃, 4.2 ml of benzyl bromide and 100 mg of NaI, and the whole is heated at 80° C. for 2 hours. Concentration to dryness is carried out and the residue is taken up in AcOEt. The organic phase is washed with H₂O, then with a saturated LiCl solution and then with a saturated NaCl solution. The organic phase is dried over MgSO₄ and concentrated to dryness. The residue is purified by chromatography over a silica column (heptane/AcOEt 95/5) to yield the title product in the form of a creamy white solid.

### Step F: (4aS,R)-3-Benzyl-2,3,4,4a,5,6-hexahydro-1H-pyrazino[1,2-a]quinolin-8-yl trifluoromethanesulphonate

A solution of 1M BBr₃/CH₂Cl₂ is added to a solution of the compound obtained in Step E (5 g) in 100 ml of CH₂Cl₂ at 0° C. and the whole is then stirred while returning gradually to ambient temperature. The temperature and stirring are maintained overnight. The mixture is then returned to 0° C. and 50 ml of MeOH are added slowly and the mixture is stirred at ambient temperature for 30 minutes. The reaction mixture is then concentrated to dryness and taken up several times with diisopropyl ether. The resulting beige crystals are then filtered off and dried. The crystals are then dissolved in 100 ml of CH₂Cl₂, and 11.6 ml of Et₃N and the triflate donor (8.84 g) are then added dropwise, and the whole is stirred at ambient temperature. Hydrolysis with H₂O is carried out and then extraction twice with CH₂Cl₂. The organic extracts are combined, dried over MgSO₄ and concentrated to dryness. The residue is then purified by chromatography over a silica column (heptane/AcOEt 9/1) to yield the title product in the form of creamy white crystals.

### Step G : Methyl (4aS,R)-3-benzyl-2,3,4,4a,5,6-hexahydro-1H-pyrazino[1,2-a]-quinoline-8-carboxylate

The compound obtained in Step F (3.6 g) is dissolved in 100 ml of a DMSO/MeOH (3/2) mixture and then there are added in succession 2.6 ml of Et₃N, 0.19 g of Pd(OAc)₂ and 0.935 g of dppf ligand. The mixture is degassed under argon for 20 minutes, then carbon monoxide is bubbled through for 30 minutes and the mixture is then saturated with carbon monoxide for 15 minutes. The whole is then hermetically sealed and heated at 65° C. for 3 hours. The mixture is allowed to cool and the carbon monoxide is removed with argon. The reaction mixture is then hydrolysd with H₂O and extracted with AcOEt. The organic extracts are combined, dried over MgSO₄ and concentrated to dryness. The residue is purified by chromatography over a silica column to yield the title product in the form of an oil which crystallises.

### Step H: Methyl (4aS,R)-2,3,4,4a,5,6-hexahydro-1H-pyrazino[1,2-a]quinoline-8-carboxylate

0.64 g of 10% Pd/C and then 2.4 g of NH₄COOH are added in succession to a solution of the compound obtained in Step G (3.2 g) in 100 ml of a mixture of THF/MeOH (50/50), and the whole is heated at 50° C. for 4 hours. The reaction mixture is then cooled and filtered, and the filtrate is concentrated to dryness. The resulting solid is taken up in diisopropyl ether, triturated, filtered and concentrated to dryness to yield the title product in the form of a white solid.

### Step I: Methyl (4aS,R)-3-[(4′-chloro-[1,1′-biphenyl]-2-yl)methyl]-2,3,4,4a,5,6-hexahydro-1H-pyrazino[1,2-a]quinoline-8-carboxylate

The compound obtained in Step H (7 g) is dissolved in 100 ml of DMF, there are then added in succession 8.85 g of K₂CO₃, 4.2 ml of 4-chloro-2′-(chloromethyl)-1,1′-biphenyl and 100 mg of NaI and the whole is heated at 80° C. for 2 hours. Concentration to dryness is carried out and the residue is then taken up in AcOEt and washed with H₂O, a saturated LiCl solution and then with a saturated NaCl solution. The organic phase is dried over MgSO₄ and concentrated to dryness. The residue is purified by chromatography over a -silica column (heptane/AcOEt 95/5) to yield the title product in the form of a creamy white-solid.

### Step J: (4aS,R)-3-[(4′-Chloro-[1,1′-biphenyl]-2-yl)methyl]-2,3,4,4a,5,6-hexahydro-1H-pyrazino[1,2-a]quinoline-8-carboxylic acid hydrochloride

9 ml of 6N HCl are added to a solution of 0.5 g of the compound obtained in Step I in 9 ml of dioxane. The whole is then heated at reflux for 4 hours and then concentrated to dryness. The resulting solid is triturated in diisopropyl ether, filtered and dried to yield the title compound in the form of a blueish white solid.

### Preparation 2: (4aS,R)-3-[(4′-Chloro-[1,1′-biphenyl]-3-yl)methyl]-2,3,4,4a,5,6-hexahydro-1H-pyrazino[1,2-a]quinoline-8-carboxylic acid

The procedure is as for Preparation 1, replacing 4-chloro-2′-(chloromethyl)-1,1′-biphenyl in Step I by 4-chloro-3′-(chloromethyl)-1,1′-biphenyl.

### Preparation 3: (4aS,R)-3-[(4′-Chloro-[1,1′-biphenyl]-4-yl)methyl]-2,3,4,4a,5,6-hexahydro-1H-pyrazino[1,2-a]quinoline-8-carboxylic acid hydrochloride

The procedure is as for Preparation 1, replacing 4-chloro-2′-(chloromethyl)-1,1′-biphenyl in Step I by 4-chloro-4′-(chloromethyl)-1,1′-biphenyl.

### Preparation 4: (4aS,R)-3-([1,1′-Biphenyl]-2-ylmethyl)-2,3,4,4a,5,6-hexahydro-1H-pyrazino[1,2-a]quinoline-8-carboxylic acid hydrochloride

The procedure is as for Preparation 1, replacing 4-chloro-2′-(chloromethyl)-1,1′-biphenyl in Step I by 2-(chloromethyl)-1,1′-biphenyl.

### Preparation 5: (4aS,R)-3-(2-Benzylbenzyl)-2,3,4,4a,5,6-hexahydro-1H-pyrazino[1,2-a]quinoline-8-carboxylic acid hydrochloride

The procedure is as for Preparation 1, replacing 4-chloro-2′-(chloromethyl)-1,1′-biphenyl in Step I by 1-benzyl-2-(chloromethyl)benzene.

### Preparation 6: (4aS,R)-3-[2-(2-Phenylethyl)benzyl]-2,3,4,4a,5,6-hexahydro-1H-pyrazino[1,2-a]quinoline-8-carboxylic acid hydrochloride

The procedure is as for Preparation 1, replacing 4-chloro-2′-(chloromethyl)-1,1′-biphenyl in Step I by 1-(chloromethyl)-2-(2-phenylethyl)benzene.

### Preparation 7: (4aS,R)-3-[(4′-Chloro-[1,1′-biphenyl]-2-yl)methyl]-2,3,4,4a,5,6-hexahydro-1H-pyrazino[1,2-a]quinoline-7-carboxylic acid hydrochloride

The procedure is as for Preparation 1, replacing 6-methoxy-2-methylquinoline in Step A by 5-methoxy-2-methylquinoline.

### Preparation 8: (4aS,R)-3-([1,1′-Biphenyl]-2-ylmethyl)-2,3,4,4a,5,6-hexahydro-1H-pyrazino[1,2-a]quinoline-7-carboxylic acid hydrochloride

The procedure is as for Preparation 1, replacing 6-methoxy-2-methylquinoline in Step A by 5-methoxy-2-methylquinoline, and replacing 4-chloro-2′-(chloromethyl)-1,1′-biphenyl in Step I by 2-(chloromethyl)-1,1′-biphenyl.

### Preparation 9: 2-([1,1′-Biphenyl]-2-ylmethyl)-1,2,3,4-tetrahydropyrazino[1,2-a]indole-8-carboxylic acid hydrochloride

### Step A: Ethyl 5-methoxy-1H-indole-2-carboxylate

14.5 ml of thionyl chloride are added dropwise using a dropping funnel to a suspension of 20 g of 5-methoxy-1H-indole-2-carboxylic acid in 100 ml of absolute ethanol at 0° C. The mixture is allowed to return gradually to ambient temperature once the addition is complete, and is then heated at gentle reflux for 4 hours. The reaction mixture is then concentrated and the resulting solid is triturated in diisopropyl ether, filtered and dried. The title product is obtained in the form of a dark brown solid.

### Step B: Ethyl 1-(cyanomethyl)-5-methoxy-1H-indole-2-carboxylate

20.8 g of the compound obtained in step A dissolved in 150 ml of anhydrous DMF are added dropwise using a dropping funnel to a suspension of 5.7 g of NaH (60%) in 100 ml of anhydrous DMF, the mixture is stirred for 30 minutes at ambient temperature and then 12 ml of chloroacetonitrile are added and the whole is stirred overnight at ambient temperature. The reaction mixture is then concentrated to dryness, taken up in AcOEt, hydrolysed with H₂O, and then extracted twice with AcOEt. The organic phases are then combined, washed with a saturated LiCl solution and then with a saturated NaCl solution, dried over MgSO₄, filtered and concentrated to dryness. The solid is then purified by chromatography over silica gel (heptane/AcOEt 95/5 ) to yield the title product in the form of a yellowish solid.

### Step C: 8-Methoxy-1,2,3,4-tetrahydropyrazino[1,2-a]indole

57 ml of a commercial 1M aluminium hydride solution in THF are added dropwise using a dropping funnel to 7.35 g of a solution of the compound obtained in Step B in 150 ml of anhydrous THF at 0° C. The mixture is allowed to return gradually to ambient temperature and is then heated at gentle reflux for 3 hours. The mixture is allowed to cool and is then returned to 0° C. and hydrolysis is carried out slowly with a saturated solution of Rochelle salt. The reaction mixture is then extracted with AcOEt. The organic extracts are combined, washed with a saturated NaCl solution, dried over MgSO₄ and concentrated to dryness. 5.7 g of a brown oil corresponding to the title product are obtained.

### Step D: 2-([1,1′-Biphenyl]-2-ylmethyl)-8-methoxy-1,2,3,4-tetrahydropyrazino[1,2-a]indole

The procedure is as for Step I of Preparation 1 starting from the compound obtained in Step C and replacing 4-chloro-2′-(chloromethyl)-1,1′-biphenyl by 2-(chloromethyl)-1,1′-biphenyl. The title product is obtained in the form of a yellowish solid.

### Step E: 2-([1,1′-Biphenyl]-2-ylmethyl)-1,2,3,4-tetrahydropyrazino[1,2-a]indol-8-yl trifluoromethanesulphonate

The procedure is as for Step F of Preparation 1. The title product is obtained in the form of an orangey yellow solid.

### Step F: Methyl 2-([1,1′-biphenyl]-2-ylmethyl)-1,2,3,4-tetrahydropyrazino[1,2-a]-indole-8-carboxylate

The procedure is as for Step G of Preparation 1. The title product is obtained in the form of a yellowish solid.

### Step G: 2-([1,1′-Biphenyl]-2-ylmethyl)-1,2,3,4-tetrahydropyrazino[1,2-a]indole-8-carboxylic acid hydrochloride

The procedure is as for Step J of Preparation 1. The title product is obtained in the form of a yellowish solid.

### Preparation 10 :2-[(4′-Chloro-[1,1′-biphenyl]-2-yl)methyl]-1,2,3,4-tetrahydro-pyrazino[1,2-a]indole8-carboxylic acid hydrochloride

The procedure is as for Preparation 9, replacing 2-(chloromethyl)-1,1′-biphenyl in Step D by 4-chloro-2′-(chloromethyl)-1,1′-biphenyl.

### Preparation 11: (10aS,R)-2-[(4′-Chloro-[1,1′-biphenyl]-2-yl)methyl]-1,2,3,4,10,10a-hexahydropyrazino[1,2-a]indole-8-carboxylic acid hydrochloride

### Step A: Methyl 2-1(4′-chloro-[1,1′-biphenyl]-2-yl)methyl]-1,2,3,4-tetrahydro-pyrazino[1,2-a]indole-8-carboxylate

The procedure is as for Preparation 9 Steps A to F, replacing 2-(chloromethyl)-1,1′-biphenyl in Step D by 4-chloro-2′-(chloromethyl)-1,1′-biphenyl.

### Step B: Methyl (10aS,R)-2-[(4′-chloro-[1,1′-biphenyl]-2-yl)methyl]-1,2,3,4,10,10a-hexahydropyrazino[1,2-a]indole-8-carboxylate

0.158 g of NaBH₃CN is added at ambient temperature to a solution of 0.2 g of the compound obtained in Step A in 5 ml of acetic acid. The reaction mixture is stirred for 48 hours. The mixture is hydrolysed with a saturated NaHCO₃ solution and then extracted with AcOEt. The organic phases are combined and then washed with a saturated NaCl solution, then dried over MgSO₄, filtered and evaporated to dryness. The title compound is then purified by chromatography over silica gel.

### Step C: (10aS,R)-2-[(4′-Chloro-[1,1′-biphenyl]-2-yl)methyl]-1,2,3,4,10,10a-hexahydropyrazino[1,2-a]indole-8-carboxylic acid hydrochloride

The procedure is as for Step J of Preparation 1 starting from the compound obtained in Step B.

### Preparation 12: (4aS)-3-[(4′-Chloro-[1,1′-biphenyl]-2-yl)methyl]-2,3,4,4a,5,6-hexahydro-1H-pyrazino[1,2-a]quinoline-8-carboxylic acid hydrochloride

### Step A: (4aS)-8-Methoxy-3-[(2S)-2-methoxy-2-phenylethanoyl]-2,3,4,4a,5,6-hexahydro-1H-pyrazino[1,2-a]quinoline

Initially, 36.5 ml of thionyl chloride are added to a solution of 4.94 g of S-methoxyphenylacetic acid in 100 ml of CH₂Cl₂. The reaction mixture is heated at 40° C. for half a day and is then allowed to cool to ambient temperature. Evaporation to dryness is carried out to obtain an oil. Secondly, the resulting oil in 120 ml of CH₂Cl₂ is added to a solution of 6.18 g of the compound obtained in Step D of Preparation 1 in 120 ml of CH₂Cl₂ and 240 ml of 1N NaOH. The whole is stirred vigorously at ambient temperature for 1 hour. The two phases are separated and extraction is carried out once with CH₂Cl₂. After washing with a saturated NaCl solution and drying over MgSO₄, concentration to dryness is carried out. The mixture is purified by flash chromatography over silica gel (petroleum ether/AcOEt 80/20) to yield a mixture of the two diastereoisomers in the form of an oil.

The diastereoisomers are then separated by optical preparative liquid chromatography over Chiralpak AD using EtOH as solvent and eluant.

### Step B: (4aS)-8-Methoxy-2,3,4,4a,5,6-hexahydro-1H-pyrazino[1,2-a]quinoline

9.6 g of KOtBu are added to a solution of 3.91 g of the compound obtained in Step A in 150 ml of THF. The reaction mixture is stirred at ambient temperature for half a day and then overnight. The THF is removed by evaporation and then the reaction mixture is hydrolysed with H₂O and extracted with AcOEt. After washing with a saturated NaCl solution and then drying over MgSO₄, concentration to dryness is carried out. The mixture is purified by flash chromatography over silica gel (CH₂Cl₂/MeOH/NH₄OH(95/5/0.5)) to yield the title product in the form of an oil.

### Step C: (4aS)-3-[(4′-Chloro-[1,1′-biphenyl]-2-yl)methyl]-2,3,4,4a,5,6-hexahydro-1H-pyrazino[1,2-a]quinoline-8-carboxylic acid hydrochloride

The procedure is as for Steps E to J of Preparation 1 starting from the compound obtained in Step B.

### Preparation 13: (4aR)-3-[(4′-Chloro-[1,1′-biphenyl]-2-yl)methyl]-2,3,4,4a,5,6-hexahydro-1H-pyrazino[1,2-a]quinoline-8-carboxylic acid hydrochloride

The procedure is as for Preparation 12, in Step A using the other diastereoisomer obtained.

### Preparation 14: (4aS)-3-([1,1′-Biphenyl]-2-ylmethyl)-2,3,4,4a,5,6-hexahydro-1H-pyrazino[1,2-a]quinoline-8-carboxylic acid hydrochloride

The procedure is as for Preparation 12, replacing 4-chloro-2′-(chloromethyl)-1,1′-biphenyl in Step I of Preparation 1 by 2-(chloromethyl)-1,1′-biphenyl.

### Preparation 15: (4aR)-3-([1,1′-Biphenyl]-2-ylmethyl)-2,3,4,4a,5,6-hexahydro-1H-pyrazino[1,2-a]quinoline-8-carboxylic acid hydrochloride

The procedure is as for Preparation 13, replacing 4-chloro-2′-(chloromethyl)-1,1′-biphenyl in Step I of Preparation 1 by 2-(chloromethyl)-1,1′-biphenyl.

### Preparation 16: (4aS,R)-3-([1,1′-Biphenyl]-2-ylsulphonyl)-2,3,4,4a,5,6-hexahydro-1H-pyrazino[1,2-a]quinoline-8-carboxylic acid hydrochloride

### Step A: 3-([1,1′-Biphenyl]-2-ylsulphonyl)-8-methoxy-2,3,4,4a,5,6-hexahydro-1H-pyrazino[1,2-a]quinoline

The procedure is as for Step E of Preparation 1, replacing benzyl bromide by [1,1′-biphenyl]-2-sulphonyl chloride.

### Step B: 3-([1,1′-Biphenyl]-2-ylsulphonyl)-2,3,4,4a,5,6-hexahydro-1H-pyrazino[1,2-a]quinoline-8-carboxylic acid hydrochloride

The procedure is as for Steps F, G and J of Preparation 1 starting from the compound obtained in Step A.

### Preparation 17 :3-[(4′-Chloro-[1,1′-biphenyl]-2-yl)methyl]-1,2,3,4,4a,5,6,7-octahydropyrazino[1,2-a][1]benzazepine-9-carboxylic acid

### Step A: 6-Methoxy-3,4-dihydro-1(2H)-naphthalenone O-methyl-oxime

28.93 g of Na₂HPO₄.2H₂O and 27.15 g of MeONH₂.HCl are added to a solution of 28.64 g of 6-methoxy-3,4-dihydro-1(2H)-naphthalenone in 500 ml of methanol. The reaction mixture is then stirred at ambient temperature for 2 hours. After concentration, the residue is taken up in a CH₂Cl₂/H₂O mixture and the organic phase is washed with water, dried over magnesium sulphate and concentrated to yield the expected product.

### Step B: N-(6-Methoxy-1,2,3,4-tetrahydro-naphthalen-1-yl)-O-methyl-hydroxylamine

17.2 ml of the complex BH3.pyridine are added to a solution of 10 g of the compound obtained in Step A in 100 ml of ethanol. Once the reaction mixture has been brought to 0° C., 200 ml of a 2.5N HCl solution are added dropwise over the course of 3 hours. The mixture is returned to ambient temperature and stirred for 1 hour, and then a saturated NaHCO₃ solution is added dropwise at 0° C. until a pH of 5 is reached. The aqueous phase is extracted with CH₂Cl₂, and then the organic phase is dried over magnesium sulphate, concentrated and purified by chromatography over a silica column (heptane/AcOEt) to yield the expected product.

### Step C: 7-Methoxy-2-vinyl-2,3,4,5-tetrahydro-1H-1-benzazepine

145 mmol of bromovinyl magnesium in 145 ml of THF are added to a solution of 10 g of the preceding compound in 100 ml of THF. The addition is carried out at 0° C. over the course of 40 minutes, and then the reaction mixture is returned to ambient temperature. After stirring for 1 hour, the mixture is hydrolysed with water dropwise at 0° C., and then the aqueous phase is extracted with CH₂Cl₂. The organic phases are combined, dried over magnesium sulphate, filtered and concentrated. The resulting residue is purified by chromatography over a silica column (heptane/AcOEt) to yield the expected product.

### Step D: tert-Butyl 7-methoxy-2-vinyl-2,3,4,5-tetrahydro-1H-1-benzazepine-1-carboxylate

2.45 g of Boc₂O and 1.55 g of K₂CO₃ are added to a solution of 1.52 g of the compound of Step C in 15 ml of THF. The reaction mixture is heated to 60° C. and stirred for 16 hours. After dilution in a mixture of AcOEt/H₂O, the aqueous phase is extracted with AcOEt, and then the organic phases are combined, washed with water and with a saturated NaCl solution, dried over magnesium sulphate, filtered and concentrated. The resulting residue is purified by chromatography over a silica column (heptane/AcOEt) to yield the expected product.

### Step E: Methyl 7-methoxy-2,3,4,5-tetrahydro-1H-1-benzazepine-2-carboxylate

12 ml of a 2.5N NaOH solution in methanol are added to a solution of 1 g of the preceding compound in 50 ml of CH₂Cl₂. The whole is cooled to −78° C. and a stream of ozone is applied. Once the characteristic blue colour has appeared, the reaction mixture is hydrolysed and extracted with AcOEt. The organic phases are combined, dried over magnesium sulphate, filtered and concentrated. The resulting residue is dissolved in a 4N HCl solution in dioxane. After neutralisation, the aqueous phase is extracted with CH₂Cl₂ and the organic phases are combined, dried over magnesium sulphate, filtered and concentrated. The resulting residue is purified by chromatography over a silica column (CH₂Cl₂/MeOH) to yield the expected product.

### Step F: Methyl 1-({[(benzyloxy)carbonyl]amino}acetyl)-7-methoxy-2,3,4,5-tetrahydro-1H-1-benzazepine-2-carboxylate

14.2 g of PCl₅ are added in portions at 0° C. to a solution of 14.3 g of [(benzyloxy)-carbonyl]aminoacetic acid in 300 ml of THF. The whole is then stirred at that same temperature for 2 hours. A solution of 10 g of the compound of Step E in 100 ml of THF and 50 ml of pyridine is added dropwise at 0° C. to the reaction mixture over the course of 2 hours. The mixture is then brought to ambient temperature and stirred for 16 hours. The resulting heterogenous mixture is hydrolysed dropwise at 0° C. and then extracted with AcOEt. The organic phases are combined, washed with a saturated NaHCO₃ and with a saturated NaCl solution, dried over magnesium sulphate, filtered and concentrated. The resulting residue is purified by chromatography over a silica column (heptane/AcOEt) to yield the expected product.

### Step G: 9-Methoxy-2,3,4a,5,6,7-hexahydropyrazino[1,2-a][1]benzazepine-1,4-dione

2 g of 10% Pd/C and 4.1 g of NH₄COOH are added in succession to a solution of 10 g of the compound obtained in Step F in 400 ml of a mixture of THF/MeOH (1/3). The whole is heated at 50° C. for 10 hours. The reaction mixture is then cooled, filtered and concentrated to dryness. The resulting solid is taken up in diisopropyl ether, triturated, filtered and concentrated to dryness to yield the expected compound.

### Step H: 3-Benzyl-9-methoxy-2,3,4a,5,6,7-hexahydropyrazino[1,2-a][1]benzazepine-1,4-dione

7 g of the compound of Step G in 450 ml of DMF at 0° C. are added dropwise over the course of 2 hours to a heterogenous solution of 1.6 g of 60% NaH in 50 ml of DMF. After returning to ambient temperature, 4 ml of benzyl bromide are added over the course of 30 minutes, and then the reaction mixture is stirred for 16 hours. After concentration, the residue is taken up at 0° C. in a mixture of AcOEt and a saturated NaHCO₃ solution. The aqueous phase is extracted with AcOEt, and then the organic phases are combined, washed with a saturated LiCl solution, dried over magnesium sulphate, filtered and concentrated. The resulting residue is purified by chromatography over a silica column (heptane/AcOEt) to yield the expected compound.

### Step I: 3-Benzyl-9-methoxy-1,2,3,4,4a,5,6,7-octahydropyrazino[1,2-a][1]-benzazepine

2.27 g of NaBH4 are added in portions at 0° C. to a solution of 7 g of the compound of Step H in 150 ml of THF. The reaction mixture is then stirred at that same temperature for 30 minutes. 11.4 ml of the complex BF₃ . Et₂O are then added dropwise at 0° C. over the course of 1 hour. After returning to ambient temperature, the reaction mixture is refluxed for 16 hours, and 50 ml of a 5N HCl solution are added dropwise at 0° C. The reaction mixture is then heated at reflux for 1 hour before being hydrolysed with 50 ml of 5N NaOH until a pH of 5 is reached. The aqueous phase is extracted with AcOEt, and then the organic phases are combined and washed with a saturated NaHCO₃ solution, dried over magnesium sulphate, filtered and concentrated. The resulting residue is purified by chromatography over a silica column (heptane/AcOEt) to yield the expected product.

### Step J: 3-Benzyl-1,2,3,4,4a,5,6,7-octahydropyrazino[1,2-a][1]benzazepin-9-yl trifluoromethanesulphonate

The procedure is as for Step F of Preparation 1.

### Step K: Methyl 3-benzyl-1,2,3,4,4a,5,6,7-octahydropyrazino[1,2-a]1[]benzazepine-9-carboxylate

The procedure is as for Step G of Preparation 1.

### Step L: Methyl 1,2,3,4,4a,5,6,7-octahydropyrazino[1,2-a][1]benzazepine-9-carboxylate hydrochloride

300 mg of palladium catalyst are added to a solution of 1.5 g of the preceding compound in 35 ml of a 1N HCl solution in methanol. The heterogenous solution is then hydrogenated for 48 hours at a pressure of 2 bars. The reaction mixture is filtered and rinsed with methanol to yield the title compound in the form of the hydrochloride.

### Step M: Methyl 3-[(4′-chloro-[1,1′-biphenyl]-2-yl)methyl]-1,2,3,4,4a,5,6,7-octahydropyrazino[1,2-a][1]benzazepine-9-carboxylate

The procedure is as for Step I of Preparation 1.

### Step N: 3-[(4′-Chloro-[1,1′-biphenyl]-2-yl)methyl]-1,2,3,4,4a,5,6,7-octahydropyrazino[1,2-a][1]benzazepine-9-carboxylic acid

387 mg of LiOH are added to a solution of 850 mg of the compound of Step M in 15 ml of a mixture of dioxane/H₂O (4/1). The whole is then stirred for 4 hours and concentrated to dryness. After dilution in 0.5N HCl, the aqueous phase is extracted with AcOEt. The organic phases are then combined, washed with a saturated NaHCO₃ solution and with a saturated NaCl solution, dried over magnesium sulphate, filtered and concentrated. The resulting solid is lyophilised in a mixture of ACN/H₂O to yield the title compound.

### Preparation 18: (10aα)-2-[(4′-Chloro-[1,1′-biphenyl]-2-yl)methyl]-1,2,3,4,10,10a-hexahydropyrazino[1,2-a]indole-8-carboxylic acid (α=R or S)

The compound is obtained by separating the racemic mixture obtained in Preparation 11 over Chiralpak AD using methanol, acetonitrile and diethylamine as eluants.

Retention time: 8,7 minutes

### Preparation 19: (10aβ)-2-[(4′-Chloro-[1,1′-biphenyl]-2-yl)methyl]-1,2,3,4,10,10a-hexahydropyrazino[1,2-a]indole-8-carboxylic acid (β=S or R)

The compound is obtained by separating the racemic mixture obtained in Preparation 11 over Chiralpak AD using methanol, acetonitrile and diethylamine as eluants.

Retention time: 9,6 minutes

### Preparation 20: 6-(tert-Butoxycarbonyl)-3-[(4′-chloro-[1,1′-biphenyl]-2-yl)methyl]-2,3,4,4a,5,6-hexahydro-1H-pyrazino[1,2-a]quinoxaline-8-carboxylic acid

### Step A: 4-[(Benzyloxy)carbonyl]-1-(2-nitrophenyl)-2-piperazinecarboxylic acid

19.5 ml of a 2.5N NaOH solution and then a solution of 2.07 g of copper sulphate in 40 ml of water are added to a solution of 5 g of 2-piperazinecarboxylic acid dihydrochloride in 20 ml of water. The resulting blue solution is cooled to 5° C., and then 2.5 g of Na₂CO₃ are added all at once, followed dropwise by a solution of 3.85 ml of benzyl chloroformate in 20 ml of dioxane. After returning to ambient temperature, the reaction mixture is stirred for 24 hours. The precipitate is removed by filtration to yield 4-[(benzyloxy)carbonyl]-2-piperazinecarboxylic acid chelated with copper. This latter compound is dissolved in 375 ml of water, and then 4.5 g of EDTA are added. The reaction mixture is heated at 80° C. for 3 hours and then concentrated to dryness. The residue is taken up in 75 ml of DMSO. There are then added 3.43 g of 2-fluoro-nitrobenzene and 15 ml of Et₃N, and then the reaction mixture is heated at 60° C. for 48 hours. After returning to ambient temperature, the solution is adjusted to pH 3 using 5N HCl and is then diluted in 250 ml of water, and extracted with AcOEt. The organic phases are washed with water, dried over magnesium sulphate, concentrated and purified by chromatography over a silica column (CH₂Cl₂/MeOH) to yield the expected product.

### Step B: Benzyl 5-oxo-1,2,4,4a,5,6-hexahydro-3H-pyrazino[1,2-a]quinoxaline-3-carboxylate

8 g of iron powder are added in portions to a solution of 5.7 g of the compound of Step A in 100 ml of acetic acid. The mixture is heated at 60° C. for 3 hours and then, after returning to ambient temperature, 50 ml of 1N HCl are added. The solution is extracted with dichloromethane and the organic phases are combined, dried over magnesium sulphate and then concentrated. The residue is purified by chromatography over a silica column (heptane/AcOEt) to yield the expected product.

### Step C: Benzyl 8-bromo-5-oxo-1,2,4,4a,5,6-hexahydro-3H-pyrazino-[1,2-a]quinoxaline-3-carboxylate

At 0° C. over a period of 25 minutes a solution of 2.55 g of N-bromosuccinimide in 30 ml of DMF is added to a solution of 4.4 g of the compound of Step B in 40 ml of DMF. The orange solution is stirred at that same temperature for 1.5 hours and is then diluted in a mixture of H₂O/AcOEt (1/1). The aqueous phase is extracted with AcOEt, and then the organic phases are combined and washed with water and then with a saturated LiCl solution before being dried over magnesium sulphate, filtered and concentrated. The resulting residue is purified by chromatography over a silica column (heptane/AcOEt) to yield the expected product.

### Step D:: Benzyl 8-bromo-1,2,4,4a,5,6-hexahydro-3H-pyrazino[1,2-a]quinoxaline-3-carboxylate

The procedure is as for Step I of Preparation 17 using the compound obtained in the preceding Step.

### Step E: 3-Benzyl 6-tert-butyl 8-bromo-4a,5-dihydro-1H-pyrazino[1,2-a]-quinoxaline-3,6(2H,4H)-dicarboxylate

The procedure is as for Step D of Preparation 17 using the compound obtained in the preceding Step.

### Step F: 3-Benzyl 6-tert-butyl 8-methyl 4a,5-dihydro-1H-pyrazino[1,2-a]-quinoxaline-3,6,8(2H,4H)-tricarboxylate

The procedure is as for Step G of Preparation 1 using the compound obtained in the preceding Step.

### Step G: 6-tert-Butyl 8-methyl 1,2,3,4,4a,5-hexahydro-6H-pyrazino[1,2-a]-quinoxaline-6,8-dicarboxylate

0.22 g of 10% Pd/C and then 144 mg of NH₄COOH are added in succession to a solution of 1.1 g of the compound obtained in Step F in 40 ml of a mixture of THF/MeOH (1/1). The whole is heated at 50° C. for 4 hours. The reaction mixture is then cooled, filtered and concentrated to dryness. The resulting solid is taken up in diisopropyl ether, triturated, filtered and concentrated to dryness to yield the title compound.

### Step H: 6-tert-Butyl 8-methyl 3-[(4′-chloro-[1,1′-biphenyl]-2-yl)methyl]-1,2,3,4,4a,5-hexahydro-6H-pyrazino[1,2-a]quinoxaline-6,8-dicarboxylate

The procedure is as for Step I of Preparation 1 using the compound obtained in the preceding Step.

### Step I: 6-(tert-Butoxycarbonyl)-3-[(4′-chloro-[1,1′-biphenyl]-2-yl)methyl]-2,3,4,4a,5,6-hexahydro-1H-pyrazino[1,2-a]quinoxaline-8-carboxylic acid

The procedure is as for Step N of Preparation 17 using the compound obtained in the preceding Step.

### Preparation 21: 6-(tert-Butoxycarbonyl)-3-[(4′-chloro-[1,1′-biphenyl]-2-yl)methyl]-5-oxo-2,3,4,4a,5,6-hexahydro-1H-pyrazino[1,2-a]quinoxaline-8-carboxylic acid

The title compound is obtained following the same Steps as for Preparation 20 but with two differences, namely: Step D is omitted whilst, in Step E, the addition of the Boc group is carried out in the presence of HNa (and not K₂CO₃) in DMF (and not THF).

### Preparation 22: 1-Bromo-2-(bromomethyl)-4,4-dimethyl-1-cyclohexene

### Step A: 4,4-Dimethyl-cyclohexanone

1 g of 5% Pd/C is added in portions to a solution of 4,4-dimethyl-2-cyclohexen-1-one (0.0805 mol, 10.6 ml) in 110 ml of AcOEt, and then the whole is stirred for 2 hours at ambient temperature under atmospheric hydrogen pressure. The palladium is removed by filtration and concentration to dryness is carried out. The resulting oil crystallises gradually. The title compound is thus obtained in the form of a white solid.

### Step B: 2-Bromo-5,5-dimethyl-1-cyclohexene-1-carbaldehyde

20.1 ml of phosphorus tribromide are added dropwise to a mixture of 110 ml of CH₂Cl₂ and 18.7 ml of DMF kept at 0° C. in an ice bath. The whole is stirred at ambient temperature for 30 minutes. The reaction mixture is then cooled to 0° C. and 10.5 g of the compound of Step A dissolved in 90 ml of CH₂Cl₂ are added. The whole is then stirred for 4 hours gradually returning to ambient temperature before being poured into a mixture of ice/saturated NaHCO₃ solution. It is then stirred for one hour and extracted with Et₂O. The organic phases are then combined, washed with a saturated NaCl solution, dried over magnesium sulphate and then concentrated to dryness The residue is then purified over a silica column (heptane AcOEt gradient 0% to 5% AcOEt). The title compound is obtained in the form of a colourless oil.

### Step C: (2-Bromo-5,5-dimethyl-1-cyclohexen-1-yl)-methanol

3.23 g of sodium borohydride are added in portions to a solution of 12.48 g of the compound of Step B at 0° C. in 120 ml of methanol. The whole is stirred for 5 hours gradually returning to ambient temperature. The reaction mixture is then cooled to 0° C., then hydrolysed and extracted with CH₂Cl₂. The organic phases are then combined, washed with a saturated NaCl solution, dried over magnesium sulphate and finally concentrated to dryness. The title compound is obtained in the form of a colourless oil, which is purified by chromatography over a silica column (heptane/AcOEt).

### Step D: 1-Bromo-2-(bromomethyl)-4,4-dimethyl-1-cyclohexene

3.96 g of the compound of Step C are dissolved in 70 ml of Et₂O, the whole being kept at 0° C. 1.7 ml of phosphorus tribromide are then added dropwise thereto. The whole is stirred at that temperature for 1 hour 30 minutes. The reaction mixture is then hydrolysed before being extracted with Et₂O. The organic phases are then combined, washed with a saturated NaHCO₃ solution and then with a saturated NaCl solution, dried over magnesium sulphate and finally concentrated to dryness. The title compound is obtained in the form of a colourless oil which is purified by chromatography over a silica column (heptane/AcOEt).

### Preparation 23: (4aR)-3-{[2-(4-Chlorophenyl)-5,5-dimethyl-1-cyclohexen-1-yl]methyl}-2,3,4,4a,5,6-hexahydro-1H-pyrazino[1,2-a]quinoline-8-carboxylic acid hydrochloride

### Step A: Methyl (4aR)-2,3,4,4a,5,6-hexahydro-1H-pyrazino[1,2-a]quinoline-8-carboxylate

The procedure is as for Steps A and B of Preparation 12, selecting the other diastereoisomer. The resulting compound is then subjected to the same treatments as those described in Steps E, F, G and H of Preparation 1.

### Step B: Methyl (4aR)-3-[(2-bromo-5,5-dimethyl-1-cyclohexen-1-yl)methyl]-2,3,4,4a,5,6-hexahydro-1H-pyrazino[1,2-a]quinoline-8-carboxylate

0.98 ml of Et₃N, 0.1 g of NaI and 1.06 g of the compound of Preparation 22 are added in succession to a solution of 0.76 g of the compound of Step A in 20 ml of DMF. The whole is then heated at 80° C. for 3 hours. After cooling to ambient temperature, the reaction mixture is hydrolysed and then extracted with AcOEt. The organic phases are combined, washed with a saturated LiCl solution and then with a saturated NaCl solution, and then dried over magnesium sulphate and concentrated to dryness. The resulting solid is then taken up in diisopropyl ether, triturated and then filtered. The title compound is obtained in the form of a white solid that is sufficiently pure to be used in the following Step.

### Step C: Methyl (4aR)-3-{[2-(4-chlorophenyl)-5,5-dimethyl-1-cyclohexen-1-yl]methyl}-2,3,4,4a,5,6-hexahydro-1H-pyrazino[1,2-a]quinoline-8-carboxylate

0.14 g of PdCl₂(Ph₃)₂, and then 0.697 g of 4-chlorophenylboronic acid and 2.4 ml of a 2M aqueous Na₂CO₃ solution are added in succession to a suspension of 1.33 g of the compound of Step B in a mixture of DME/H₂O/EtOH (15 ml/6 ml/4 ml). The whole is degassed under argon for 15 minutes and then heated at 80° C. for 16 hours. The reaction mixture is then filtered at ambient temperature. The filtrate is then hydrolysed and extracted with CH₂Cl₂. The organic phases are combined, washed with a saturated NaCl solution and then dried over magnesium sulphate and concentrated to dryness. The resulting green oil is then purified by chromatography over silica gel (heptane/AcOEt: 95/5) to yield the title compound in the form of a white solid.

### Step D: (4aR)-3-([2-(4-Chlorophenyl)-5,5-dimethyl-1-cyclohexen-1-yl]methyl}-2,3,4,4a,5,6-hexahydro-1H-pyrazino[1,2-a]quinoline-8-carboxylic acid hydrochloride

A suspension of 0.910 g of the compound of Step C in a mixture of dioxane/ 6N HCl (10 ml/15 ml) is heated at 70° C. for 20 hours. The mixture is then allowed to return to ambient temperature. The resulting precipitate is filtered off and then washed with diisopropyl ether and dried. The title compound is obtained in the form of a light green solid.

### Preparation 24: (10aβ)-2-{[2-(4-Chlorophenyl)-5,5-dimethyl-1-cyclohexen-1-yl]methyl}-1,2,3,4,10,10a-hexahydropyrazino[1,2-a]indole-8-carboxylic acid (β=S or R)

### Step A: 2-{[2-(4-Chlorophenyl)-5,5-dimethyl-1-cyclohexen-1-yl]methyl}-8-methoxy-1,2,3,4-tetrahydropyrazino[1,2-a]indole

The compound obtained in Step C of Preparation 9 is subjected to the procedures described in Steps B and C of Preparation 23.

### Step B: Methyl 2-{[2-(4-chlorophenyl)-5,5-dimethyl-1-cyclohexen-1-yl]methyl)-1,2,3,4-tetrahydropyrazino[1,2-a]indole-8-carboxylate

The compound of Step A is subjected to the procedures of Steps E and F of Preparation 9.

### Step C: 2-{[2-(4-Chlorophenyl)-5,5-dimethyl-1-cyclohexen-1-yl]methyl}-1,2,3,4,10,10a-hexahydropyrazino[1,2-a]indole-8-carboxylic acid hydrochloride

The compound of the preceding Step is subjected to the procedures of Steps B and C of Preparation 11.

### Step D: (10aβ)-2-{[2-(4-Chlorophenyl)-5,5-dimethyl-1-cyclohexen-1-yl]methyl}-1,2,3,4,10,10a-hexahydropyrazino[1,2-a]indole-8-carboxylic acid (β=S or R)

The compound is obtained by separating the mixture of enantiomers obtained in Step C.

### Preparation 25: (4aR)-3-{[4-(4-Chlorophenyl)-3-pyridyl]methyl}-2,3,4,4a,5,6-hexahydro-1H-pyrazino[1,2-a]quinoline-8-carboxylic acid trifluoroacetate

### Step A: (4-Bromo-3-pyridyl)methanol

0.2 g of sodium borohydride is added in portions to a solution of 1 g of 4-bromonicotinaldehyde in 50 ml of MeOH at 0° C. The whole is then stirred for 6 hours gradually returning to ambient temperature. The reaction mixture is cooled to 0° C., then hydrolysed with a saturated NH₄Cl solution and extracted with CH₂Cl₂. The organic phases are then combined, washed with a saturated NaCl solution, dried over magnesium sulphate and then concentrated to dryness. The title compound is obtained in the form of a light brown gel which is used as is in the following Step.

### Step B: [4-(4-Chlorophenyl)-3-pyridyl]methanol

0.335 g of Pd(Ph₃)₄, 0.453 g of 4-chlorophenylboronic acid and then 2.9 ml of a 2M aqueous Na₂CO₃ solution are added in succession to a suspension of 0.545 g of the compound of Step A in a mixture of DME/EtOH (7.5 ml/3 ml). The whole is degassed under argon for 15 minutes and then heated at 80° C. for 18 hours. The reaction mixture is then filtered at ambient temperature. The filtrate is then hydrolysed and extracted with CH₂Cl₂. The organic phases are combined, washed with a saturated NaCl solution and then dried over magnesium sulphate and concentrated to dryness. The resulting solid is finally purified by chromatography over silica gel (CH₂Cl₂/MeOH ) to yield the title compound.

### Step C: 3-(Chloromethyl)-4-(4-chlorophenyl)pyridine

A solution of 0.590 ml of thionyl chloride (0.008 mol) in 5 ml of CH₂Cl₂ is added dropwise to a solution of 0.176 g of the compound of Step B in 5 ml of CH₂Cl₂ at 0° C. The whole is stirred for 2 hours gradually returning to ambient temperature. The reaction mixture is then concentrated to dryness. The resulting solid is washed with heptane and dried. The title compound is obtained in the form of a light beige solid which is used as is in the following Step.

### Step D: Methyl (4aR)-3-{[4-(4-chlorophenyl)-3-pyridyl]methyl}-2,3,4,4a,5,6-hexahydro-1H-pyrazino[1,2-a]quinoline-8-carboxylate

The procedure is as for Step B of Preparation 23, reacting the compound of Step A of Preparation 23 with the compound of preceding Step C.

### Step E: (4aR)-3-{[4-(4-Chlorophenyl)-3-pyridyl]methyl}-2,3,4,4a,5,6-hexahydro-1H-pyrazino[1,2-a]quinoline-8-carboxylic acid trifluoroacetate

The preceding compound is subjected to the procedure of Step N of Preparation 17. A non-crystalline compound is obtained, which is purified by inverse phase chromatography (C-18) (gradient H₂O, CH₃CN, 0.1% of TFA). After lyophilisation, the title product is obtained in the form of a TFA salt.

### Preparation 26: (4aR)-3-{[2-(4-Chlorophenyl)-3-pyridyl]methyl}-2,3,4,4a,5,6-hexahydro-1H-pyrazino[1,2-a]quinoline-8-carboxylic acid trifluoroacetate

The procedure is as for Preparation 25, replacing 4-bromonicotinaldehyde by 2-bromo-nicotinaldehyde.

### Preparation 27: (4aR)-3-{[3-(4-Chlorophenyl)-2-pyridyl]methyl}-2,3,4,4a,5,6-hexahydro-1H-pyrazino[1,2-a]quinoline-8-carboxylic acid trifluoroacetate

The procedure is as for Preparation 25, replacing 4-bromonicotinaldehyde by 3-bromo-2-pyridinecarbaldehyde.

### Preparation 28: (4aR)-3-{[3-(4-Chlorophenyl)-4-pyridyl]methyl}-2,3,4,4a,5,6-hexahydro-1H-pyrazino[1,2-a]quinoline-8-carboxylic acid trifluoroacetate

The procedure is as for Preparation 25, replacing 4-bromonicotinaldehyde by 3-bromo-isonicotinaldehyde.

### Preparation 29: (4aR)-3-[(4-[(tert-Butoxycarbonyl)amino]-4′-chloro-[1,1′-biphenyl]-2-yl)methyl]-2,3,4,4a,5,6-hexahydro-1H-pyrazino[1,2-a]quinoline-8-carboxylic acid

### Step A: Methyl 4-nitro-4′-chloro-[1,1′-biphenyl]-2-carboxylate

This compound is obtained using the coupling method described in Step B of Preparation 25, replacing (4-bromo-3-pyridyl)methanol by methyl 2-bromo-5-nitrobenzoate. The expected product is obtained after a purification step over silica gel (petroleum ether/AcOEt) in the form of a yellow solid.

### Step B: 4-Nitro-4′-chloro-[1,1′-biphen-2-yl]methanol

0.617 g of sodium borohydride is added in portions to a solution of 2.38 g of the compound of Step A in 20 ml of MeOH at 0° C. The whole is stirred for 6 hours gradually returning to ambient temperature, and then heated at reflux for 24 hours. The reaction mixture is then cooled to 0° C., hydrolysed with a saturated NH₄Cl solution and extracted with CH₂Cl₂. The organic phases are then combined and washed with a saturated NaCl solution, dried over magnesium sulphate and then concentrated to dryness. After purification over silica gel (petroleum ether/AcOEt), the expected compound is obtained in the form of a yellow solid.

### Step C: 4-Amino-4′-chloro-[1,1′-biphen-2-yl]methanol

3.8 g of stannic chloride (SnCl₂) are added in portions to a solution of 0.890 g of the compound of Step B in a mixture of THF (15 ml)/MeOH (20 ml). The whole is stirred gradually at reflux for 3 hours. The reaction mixture is then concentrated to dryness, taken up in CH₂Cl₂, cooled to 0° C. before being hydrolyed with a 5N NaOH solution and extracted with CH₂Cl₂. The organic phases are then combined, washed with a saturated NaCl solution, dried over magnesium sulphate and then concentrated to dryness. The expected compound is obtained in the form of a yellow solid used as is in the following Step.

### Step D: 4-[(tert-Butoxycarbonyl)amino]-4′-chloro-2-(hydroxymethyl)-1,1′-biphenyl

0.71 g of Boc₂O is added to a solution of 0.76 g of the compound of Step C in 25 ml of ethanol. The whole is stirred for 20 hours gradually going to 35° C. The reaction mixture is then concentrated to dryness, taken up in Et₂O, then hydrolysed and extracted with Et₂O. The organic phases are then combined, washed with a saturated NaCl solution, dried over magnesium sulphate and then concentrated to dryness. After purification over silica gel (petroleum ether/AcOEt), the title compound is obtained in the form of a beige solid.

### Step E: 4-[(tert-Butoxycarbonyl)amino]-4′-chloro-2-(chloromethyl)-1,1′-biphenyl

0.532 ml of Et₃N and 0.22 ml of mesyl chloride (0.00284 mol) are added in succession to a solution of 0.634 g of the compound of Step D in 15 ml of THF at 0° C. The whole is then stirred for 96 hours gradually returning to ambient temperature. The reaction mixture is then concentrated to dryness. After purification over silica gel (petroleum ether/AcOEt), the expected compound is obtained in the form of a yellow oil which crystallises.

### Step F: Methyl (4aR)-3-({4-[(tert-butoxycarbonyl)amino]-4′-chloro-[1,1′-biphenyl]-2-yl) methyl)-2,3,4,4a,5,6-hexahydro-1H-pyrazino[1,2-a]quinoline-8-carboxylate

The procedure is as for Step B of Preparation 23, reacting the compound of Step A of Preparation 23 with the compound of the preceding Step E.

### Step G: (4aR)-3-[(4-[(tert-Butoxycarbonyl)amino]-4′-chloro-[1,1′-biphenyl]-2-yl)methyl]-2,3,4,4a,5,6-hexahydro-1H-pyrazino[1,2-a]quinoline-8-carboxylic acid

The preceding compound is subjected to the procedure of Step N of Preparation 17.

### Preparation 30: (4aR)-3-[(4-{[(tert-Butoxycarbonyl)amino]methyl}-4′-chloro-[1,1′-biphenyl]-2-yl)methyl]-2,3,4,4a,5,6-hexahydro-1H-pyrazino[1,2-a]quinoline-8-carboxylic acid

### Step A: Methyl 4-methoxy-4′-chloro-[1,1′-biphenyl]-2-carboxylate

This compound is obtained using the coupling method described in Step B of Preparation 25, replacing (4-bromo-3-pyridyl)methanol by methyl 2-bromo-5-methoxybenzoate. The expected product is obtained in the form of a solid after a purification step over silica gel (heptane/AcOEt).

### Step B: Methyl 4′-chloro-4-hydroxy-[1,1′-biphenyl]-2-carboxylate

A 1M solution of BBr₃ in 42 ml of CH₂Cl₂ is added slowly to a solution of 1.6 g of the compound of Step A in 20 ml of CH₂Cl₂ at −78° C. The whole is stirred at that temperature for 1 hour 30 minutes. A mixture of H₂O/MeOH (40 ml/10 ml) is then added. The whole is stirred for 45 minutes still at −78° C., and is then extracted with CH₂Cl₂. The organic phases are then combined and dried over magnesium sulphate before being concentrated to dryness. A brown foam is obtained, which is used as is in the following step.

### Step C: Methyl 4′-chloro-4-trifluoromethanesulphonyl-[1,1′-biphenyl]-2-carboxylate

4 ml of Et₃N (0.029 mol) and 3.1 g of N-phenyl-bis(trifluoromethanesulphonamide) are added in succession to a solution of 1.5 g of the compound of Step B in 20 ml of CH₂Cl₂ at 0° C. The whole is then stirred for 20 hours gradually returning to ambient temperature. The reaction mixture is then concentrated to dryness, taken up in AcOEt and washed, in succession, with a 1N HCl solution, a saturated NaHCO₃ solution and a saturated NaCl solution, and is then dried over magnesium sulphate. After purification over silica gel (heptane/AcOEt), the expected compound is obtained in the form of a colourless oil.

### Step D: Methyl 4′-chloro-4-cyano-[1,1′-biphenyl]-2-carboxylate

0.44 g of Pd₂(dba)₃, 0.066 g of dppf and 0.422 g of Zn(CN)₂ are added in succession to a solution of 1.2 g of the compound of Step C in 50 ml of DMF. The whole is then stirred for 3 hours gradually going to 90° C. The reaction mixture is then concentrated, taken up in AcOEt and washed, in succession, with a saturated LiCl solution and a saturated NaCl solution before being dried over magnesium sulphate. After purification over silica gel (heptane/AcOEt), the expected compound is obtained in the form of a colourless oil which crystallises gradually.

### Step E: Methyl 4-aminomethyl-4′-chloro-[1,1′-biphenyl]-2-carboxylate

1.68 g of NiCl₂ and 1.47 g of sodium borohydride are added in portions to a suspension of 3.52 g of the compound of Step D in 40 ml of MeOH at 0° C. The whole is then stirred for 6 hours gradually returning to ambient temperature. The reaction mixture is then filtered, and the filtrate is diluted with AcOEt before being hydrolysed. The organic phases are then combined, washed with a saturated NaCl solution, dried over magnesium sulphate and then concentrated. The title compound is obtained in the form of a white foam which is used as is in the following step.

### Step F: Methyl 4-{[(tert-butoxycarbonyl)amino]methyl}-4′-chloro-[1,1′-biphenyl]-2-carboxylate

2.82 g of Boc₂O are added to a solution of 3.02 g of the compound of Step E in 60 ml of CH₂Cl₂. The whole is then stirred at ambient temperature for 20 hours. The reaction mixture is then concentrated to dryness. After purification over silica gel (petroleum ether/AcOEt), the title compound is obtained in the form of a white solid.

### Step G: 4-{[(tert-Butoxycarbonyl)amino]methyl}-4′-chloro-2-(hydroxymethyl)-1,1′-biphenyl

A 2.4M solution of LAH in THF is added dropwise to a solution of 1.6 g of the compound of Step F at 0° C. in 60 ml of THF. The whole is stirred at ambient temperature for 2 hours. The reaction mixture is then hydrolysed with a saturated solution of Rochelle salt at ambient temperature for 1 hour 30 minutes. It is then extracted with AcOEt. The organic extracts are then combined, washed with a saturated NaCl solution and dried over magnesium sulphate, and then concentrated to dryness. After purification over silica gel (petroleum ether/AcOEt), the title compound is obtained in the form of a translucent oil.

### Step H: 4-{[(tert-Butoxycarbonyl)amino]methyl}-4′-chloro-2-(chloromethyl)-1,1′-biphenyl

1.26 ml of Et₃N (0.00896 mol) and 0.52 ml of mesyl chloride (0.00672 mol) are added in succession to a solution of 1.56 g of the compound of Step G in 50 ml of THF at 0° C. The whole is then stirred for 96 hours gradually returning to ambient temperature. The reaction mixture is then concentrated to dryness. After purification over silica gel (petroleum ether/AcOEt), the expected compound is obtained in the form of an oil which crystallises.

### Step I: Methyl (4aR)-3-1(4-{[(tert-butoxycarbonyl)amino]methyl}-4′-chloro-[1,1′-biphenyl]-2-yl)methyl]-2,3,4,4a,5,6-hexahydro-1H-pyrazino[1,2-a]quinoline-8-carboxylate

The procedure is as for Step B of Preparation 23, reacting the compound of Step A of Preparation 23 with the compound of the preceding Step H. The title compound is obtained after a purification step over silica gel (heptane/AcOEt).

### Step J: (4aR)-3-[(4-{[(tert-Butoxycarbonyl)amino]methyl}-4′-chloro-[1,1′-biphenyl]-2-yl)methyl]-2,3,4,4a,5,6-hexahydro-1H-pyrazino[1,2-a]quinoline-8-carboxylic acid

The preceding compound is subjected to the procedure of Step N of Preparation 17. The expected product is obtained in the form of a white solid.

### Preparation 31: (4aR)-3-[(3′-Fluoro-4′-chloro-[1,1′-biphenyl]-2-yl)methyl]-2,3,4,4a,5,6-hexahydro-1H-pyrazino[1,2-a]quinoline-8-carboxylic acid

### Step A: Methyl (4aR)-3-(2-bromobenzyl)-2,3,4,4a,5,6-hexahydro-1H-pyrazino[1,2-a]quinoline-8-carboxylate

The procedure is as for Step B of Preparation 23, reacting the compound of Step A of Preparation 23 with 1-bromo-2-(bromomethyl)benzene.

### Step B: Methyl (4aR)-3-[(3′-fluoro-4′-chloro-[1,1′-biphenyl]-2-yl)methyl]-2,3,4,4a,5,6-hexahydro-1H-pyrazino[1,2-a]quinoline-8-carboxylate

The procedure is as for Step B of Preparation 25, replacing 4-chlorophenylboronic acid by 3-fluoro-4-chlorophenylboronic acid.

### Step C: (4aR)-3-[(3′-Fluoro-4′-chloro-[1,1′-biphenyl]-2-yl)methyl]-2,3,4,4a,5,6-hexahydro-1H-pyrazino[1,2-a]quinoline-8-carboxylic acid

The preceding compound is subjected to the procedure of Step N of Preparation 17.

### Preparation 32: (4aR)-3-[(4′-Cyano-[1,1′-biphenyl]-2-yl)methyl]-2,3,4,4a,5,6-hexahydro-1H-pyrazino[1,2-a]quinoline-8-carboxylic acid

The procedure is as for Preparation 31, replacing 3-fluoro-4-chlorophenylboronic acid in Step B by 4-cyanoboronic acid.

### Preparation 33: (4aR)-3-[(4′-Trifluoro-[1,1′-biphenyl]-2-yl)methyl]-2,3,4,4a,5,6-hexahydro-1H-pyrazino[1,2-a]quinoline-8-carboxylic acid

The procedure is as for Preparation 31, replacing 3-fluoro-4-chlorophenylboronic acid in Step B by 4-trifluoromethylboronic acid.

### Preparation 34: (4aR)-3-[2-(1,3-Benzodioxol-5-yl)benzyl]-2,3,4,4a,5,6-hexahydro-1H-pyrazino[1,2-a]quinoline-8-carboxylic acid

The procedure is as for Preparation 31, replacing 3-fluoro-4-chlorophenylboronic acid in Step B by 1,3-benzodioxol-5-ylboronic acid.

### Preparation 35: (4aR)-3-Benzhydryl-2,3,4,4a,5,6-hexahydro-1H-pyrazino[1,2-a]quinoline-8-carboxylic acid

### Step A: Methyl (4aR)-3-benzhydryl-2,3,4,4a,5,6-hexahydro-1H-pyrazino[1,2-a]quinoline-8-carboxylate

The procedure is as for Step B of Preparation 23, reacting the compound of Step A of Preparation 23 with [bromo(phenyl)methyl]benzene.

### Step B: (4aR)-3-Benzhydryl-2,3,4,4a,5,6-hexahydro-1H-pyrazino[1,2-a]-quinoline-8-carboxylic acid

The preceding compound is subjected to the procedure of Step N of Preparation 17.

### Preparation 36: (4aS,R)-3-{[4-(4-Chlorophenyl)-3-pyridyl]methyl}-2,3,4,4a,5,6-hexahydro-1H-pyrazino[1,2-a]quinoline-8-carboxylic acid trifluoroacetate

The procedure is as for Preparation 25, in Step D replacing the compound of Step A of Preparation 23 by the enantiomer mixture of Step H of Preparation 1.

### Preparation 37: (4aS,R)-3-{[2-(4-Chlorophenyl)-3-pyridyl]methyl}-2,3,4,4a,5,6-hexahydro-1H-pyrazino[1,2-a]quinoline-8-carboxylic acid trifluoroacetate

The procedure is as for Preparation 26, in Step D replacing the compound of Step A of Preparation 23 by the compound of Step H of Preparation 1.

### Preparation 38: (4aS,R)-3-{[3-(4-Chlorophenyl)-2-pyridyl]methyl}-2,3,4,4a,5,6-hexahydro-1H-pyrazino[1,2-a]quinoline-8-carboxylic acid trifluoroacetate

The procedure is as for Preparation 27, in Step D replacing the compound of Step A of Preparation 23 by the compound of Step H of Preparation 1.

### Preparation 39: (4aS,R)-3-{[3-(4-Chlorophenyl)-4-pyridyl]methyl}-2,3,4,4a,5,6-hexahydro-1H-pyrazino[1,2-a]quinoline-8-carboxylic acid trifluoroacetate

The procedure is as for Preparation 28, in Step D replacing the compound of Step A of Preparation 23 by the compound of Step H of Preparation 1.

### Preparation 40: (4aS,R)-3-[(2-(4-Pyridyl)-3-pyridyl)methyl]-2,3,4,4a,5,6-hexahydro-1H-pyrazino[1,2-a]quinoline-8-carboxylic acid trifluoroacetate

The procedure is as for Preparation 25, using 2-bromo-nicotinaldehyde in Step A, 4-pyridylboronic acid in Step B and, in Step D, using the mixture of enantiomers of Step H of Preparation 1 as tricyclic synthon.

### Preparation 41: (4aS,R)-3-[(2-(6-Chloro-pyrid-3-yl)-3-pyridyl)methyl]-2,3,4,4a,5,6-hexahydro-1H-pyrazino[1,2-a]quinoline-8-carboxylic acid trifluoroacetate

The procedure is as for Preparation 25, using 2-bromo-nicotinaldehyde in Step A, 6-chloro-3-pyridylboronic acid in Step B and, in Step D, using the mixture of enantiomers of Step H of Preparation 1 as tricyclic synthon.

### Preparation 42: (4aS,R)-3-[(2-(6-Hydroxy-pyrid-3-yl)-3-pyridyl)methyl]-2,3,4,4a,5,6-hexahydro-1H-pyrazino[1,2-a]quinoline-8-carboxylic acid trifluoroacetate

The procedure is as for Preparation 25, using 2-bromo-nicotinaldehyde in Step A, 6-hydroxy-3-pyridylboronic acid in Step B and, in Step D, using the mixture of enantiomers of Step H of Preparation 1 as tricyclic synthon.

### Preparation 43: 2-{[2-(4-Chlorophenyl)-3-pyridyl]methyl}-1,2,3,4-tetrahydro-pyrazino[1,2-a]indole-8-carboxylic acid trifluoroacetate

The procedure is as for Preparation 25, using 2-bromo-nicotinaldehyde in Step A, 4-chlorophenylboronic acid in Step B and, in Step D, using the compound of Step F of Preparation 9 as tricyclic synthon.

### Preparation 44: 2-{[2-(6-Chloro-pyrid-3-yl)-3-pyridyl]methyl}-1,2,3,4-tetrahydropyrazino[1,2-a]indole-8-carboxylic acid trifluoroacetate

The procedure is as for Preparation 25, using 2-bromo-nicotinaldehyde in Step A, 6-chloro-3-pyridylboronic acid in Step B and, in Step D, using the compound of Step F of Preparation 9 as tricyclic synthon.

### Preparation 45: (4aS,R)-3-[(4′-tert-Butyl-[1,1′-biphenyl]-2-yl)methyl]-2,3,4,4a,5,6-hexahydro-1H-pyrazino[1,2-a]quinoline-8-carboxylic acid

The procedure is as for Preparation 31, replacing 3-fluoro-4-chlorophenylboronic acid in Step B by 4-tert-butyl-phenylboronic acid, and in Step C using the mixture methyl (4aS,R)-2,3,4,4a,5,6-hexahydro-1H-pyrazino[1,2-a]quinoline-8-carboxylate (Step H of Preparation 1) instead of methyl (4aR)-2,3,4,4a,5,6-hexahydro-1H-pyrazino[1,2-a]quinoline-8-carboxylate (Step A of Preparation 23).

### Preparation 46: (4aS,R)-3-[2-(4-Chlorobenzyl)benzyl]-2,3,4,4a,5,6-hexahydro-1H-pyrazino[1,2-a]quinoline-8-carboxylic acid

The procedure is as for Preparation 1, in Step I replacing 4-chloro-2′-(chloromethyl)-1,1′-biphenyl by 1-chloro-2-(4-chlorobenzyl)benzene.

### Preparation 47: 3-(2-Phenoxybenzyl)-2,3,4,4a,5,6-hexahydro-1H-pyrazino[1,2-a]quinoline-8-carboxylic acid

### Step A: Methyl 3-(2-phenoxybenzyl)-2,3,4,4a,5,6-hexahydro-1H-pyrazino[1,2-a]quinoline-8-carboxylate

The compound of Step H of Preparation 1 is subjected to reductive amination by reacting it with 2-phenoxybenzaldehyde in the presence of NaBH(OAC)₃. The reaction mixture is then treated with acetic acid and then extracted with CH₂Cl₂.

### Step B: 3-(2-Phenoxybenzyl)-2,3,4,4a,5,6-hexahydro-1H-pyrazino[1,2-a]quinoline-8-carboxylic acid

The procedure is as for Step J of Preparation 1.

### EXAMPLE 1

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{(1R)-3-(dimethylamino)-1-[(phenylsulphanyl)methyl]propyl}amino)-3-nitrobenzenesulphonamide bistrifluoroacetate

### Step A: N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide

2.05 ml of DIEA and then 1.5 g of 4-({(1R)-3-(dimethylamino)-1-[(phenylsulphanyl)methyl]propyl}amino)-3-nitrobenzenesulphonamide and then 0.783 g of EDC and 0.5 g of DMAP are added at ambient temperature to a solution of 1.26 g of the compound obtained in Preparation 1 in 50 ml of a mixture of CH₂Cl₂/THF(1/1). The reaction mixture is stirred at ambient temperature for 2 days. Evaporation to dryness is carried out and then the resulting residue is taken up in a saturated NH₄Cl solution and extraction is carried out twice with CH₂Cl₂ The organic phase is washed with a saturated NaCl solution and then dried over MgSO₄, filtration is carried out and evaporation to dryness. The resulting oil is purified by flash chromatography over silica gel (CH₂Cl₂/MeOH/NH OH 84/16/1.6) and then lyophilised to yield the title product in the form of a yellow solid.

### Elemental Microanalysis:

| | % C | % H | % N | % S |
|---|---|---|---|---|
| Calculated | 62.95 | 5.64 | 10.01 | 7.64 |
| Found | 63.20 | 5.62 | 9.78 | 7.22 |

### Step B: N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide bistrifluoroacetate

The compound obtained in Step A (0.3 g) is dissolved in 10 ml of CH₂Cl₂ at 0° C., and then trifluoroacetic acid (56 μl) is added dropwise. The whole is then stirred at ambient temperature for 30 minutes and then concentrated to dryness. The resulting solid is then taken up in H₂O and CH₃CN is added dropwise until complete dissolution of the reaction mixture, which is then lyophilised at low temperature for 24 hours. A cotton wool-like yellow solid is obtained corresponding to the title product.

### EXAMPLE 2

### N-({(4aS,R)-3-[(4′-Chloro-[1,1′-biphenyl]-3-yl)methyl]-2,3,4,4a,5,6-hexahydro-1H-pyrazino[1,2-a]quinolin-8-yl}carbonyl)-3-nitro4-{[2-(phenylsulphanyl)ethyl]amino}benzenesulphonamide hydrochloride

### Step A: N-({(4aS,R)-3-[(4′-Chloro-[1,1′-biphenyl]-3-yl)methyl]-2,3,4,4a,5,6-hexahydro-1H-pyrazino [1,2-a]quinolin-8-yl}carbonyl)-3-nitro-4-{[2-(phenylsulphanyl)ethyl]amino}benzenesulphonamide

The procedure is as for Step A of Example 1, replacing the compound of Preparation 1 by the compound of Preparation 2, and replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Step B: N-({(4aS,R)-3-[(4′-Chloro-[1,1′-biphenyl]-3-yl)methyl]-2,3,4,4a,5,6-hexahydro-1H-pyrazino [1,2-a]quinolin-8-yl]carbonyl)-3-nitro-4-{[2-(phenylsulphanyl)ethyl]amino)benzenesulphonamide hydrochloride

The compound obtained in Step A (0.3 g) is dissolved in 10 ml of CH₂Cl₂ at 0° C., and then a solution of hydrochloric acid in Et₂O (2M) (375 μl) is added dropwise. The whole is then stirred at ambient temperature for 30 minutes, and then concentrated to dryness. The resulting solid is then taken up in H₂O and CH₃CN is added dropwise until complete dissolution of the reaction mixture, which is then lyophilised at low temperature for 24 hours.

### EXAMPLE 3

### N-({(4aS,R)-3-[(4′-Chloro-[1,1′-biphenyl]-4-yl)methyl]-2,3,4,4a,5,6-hexahydro-1H-pyrazino[1,2-a]quinolin-8-yl)carbonyl)-3-nitro-4-{[2-(phenylsulphanyl)ethyl]amino}benzenesulphonamide hydrochloride

### Step A: N-({(4aS,R)-3-[(4′-Chloro-[1,1′-biphenyl]-4-yl)methyl]-2,3,4,4a,5,6-hexahydro-1H-pyrazino[1,2-a]quinolin-8-yl}carbonyl)-3-nitro-4-{[2-(phenylsulphanyl)ethyl]amino}benzenesulphonamide

The procedure is as for Step A of Example 1, replacing the compound of Preparation 1 by the compound of Preparation 3, and replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Step B: N-({(4aS,R)-3-[(4′-Chloro-[1,1′-biphenyl]-4-yl)methyl]-2,3,4,4a,5,6-hexahydro-1H-pyrazino[1,2-a]quinolin-8-yl}carbonyl)-3-nitro-4-{[2-(phenylsulphanyl)ethyl]amino}benzenesulphonamide hydrochloride

The procedure is as for Step B of Example 2.

### EXAMPLE 4

### N-{[(4aS,R)-3-[1,1′-Biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)methyl]propyl}amino)-3-nitrobenzenesulphonamide bis(hydrochloride)

### Step A: N-{[(4aS,R)-3-([1,1′-Biphenyl]-2-ylmethyl)-2,3,4,4a,5,6-hexahydro-1H-pyrazino[1,2-a]quinolin-8-yl]carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 4.

### Elemental Microanalysis:

| | % C | % H | % N | % S |
|---|---|---|---|---|
| Calculated | 65.65 | 6.01 | 10.44 | 7.97 |
| Found | 65.24 | 5.94 | 10.24 | 7.89 |

### Step B: N-{[(4aS,R)-3-([1,1′-Biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)methyl]propyl) amino)-3-nitrobenzenesulphonamide bis(hydrochloride)

The procedure is as for Step B of Example 2.

### Elemental Microanalysis:

| | % C | % H | % N | % S |
|---|---|---|---|---|
| Calculated | 59.21 | 5.69 | 9.42 | 7.19 |
| Found | 58.54 | 5.92 | 9.06 | 6.50 |

### EXAMPLE 5

### N-{[(4aS,R)-3-2-Benzylbenzyl)-2,3,4,4a,5,6-hexahydro-1H-pyrazino[1,2-a]quinolin-8-yl]carbonyl}-3-nitro-4-{[2-(phenyl-sulphanyl)ethyl]amino}benzenesulphonamide hydrochloride

### Step A: N-{[(4aS,R)-3-(2-Benzylbenzyl)-2,3,4,4a,5,6-hexahydro-1H-pyrazino[1,2-a]quinolin-8-yl]carbonyl}-3-nitro-4-{[2-(phenylsulphanyl)ethyl]-amino}benzenesulphonamide

The procedure is as for Step A of Example 1, replacing the compound of Preparation 1 by the compound of Preparation 5, and replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Step B: N-{[(4aS,R)-3-(2-Benzylbenzyl)-2,3,4,4a,5,6-hexahydro-1H-pyrazino[1,2-a]quinolin-8-yl]carbonyl}-3-nitro-4-{[2-(phenylsulphanyl)ethyl]-amino}benzenesulphonamide hydrochloride

The procedure is as for Step B of Example 2.

### EXAMPLE 6

### 3-Nitro-N-{(4aS,R-3-[2-(2-phenylethyl)benzyl]-2,3,4,4a,5,6-hexahydro-1H-pyrazino[1,2-a]quinolin-8-yl}carbonyl)-4-{[2-(phenylsulphanyl)-ethyl]amino}benzenesulphonamide

The procedure is as for Step A of Example 1, replacing the compound of Preparation 1 by the compound of Preparation 6, and replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Elemental Microanalysis:

| | % C | % H | % N | % S |
|---|---|---|---|---|
| Calculated | 66.21 | 5.69 | 9.19 | 8.42 |
| Found | 65.98 | 5.93 | 8.89 | 8.08 |

### EXAMPLE 7

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-3-nitro4-{[2-(phenylsulphanyl)ethyl]amino}benzenesulphonamide

The procedure is as for Step A of Example 1, replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Elemental Microanalysis

| | % C | % H | % N | % S |
|---|---|---|---|---|
| Calculated | 62.53 | 4.98 | 9.11 | 8.35 |
| Found | 62.53 | 5.12 | 8.70 | 8.12 |

### EXAMPLE 8

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl)carbonyl)-3-nitro-4-[(2-phenoxyethyl)amino]benzenesulphonamide

The procedure is as for Step A of Example 1, replacing 4-({(1R)-3-(dimethylamino)-1-[(phenylsulphanyl)methyl]propyl}amino)-3-nitrobenzenesulphonamide by 3-nitro-4-[(2-phenoxyethyl)amino]benzenesulphonamide.

### Elemental Microanalysis:

| | % C | % H | % N | % S |
|---|---|---|---|---|
| Calculated | 63.86 | 5.09 | 9.31 | 4.26 |
| Found | 63.82 | 5.23 | 8.98 | 3.82 |

### EXAMPLE 9

### N-{[(4aS,R)-3-([1,1′-Biphenyl]-2-ylmethyl)-2,3,4,4a,5,6-hexahydro-1H-pyrazino[1,2-a]quinolin-8-yl]carbonyl)-3-nitro-4-[(3-phenylpropyl)-amino]benzenesulphonamide

The procedure is as for Step A of Example 1, replacing the compound obtained in Preparation 1 by the compound obtained in Preparation 4, and replacing 4-({(1R)-3-(dimethylamino)-1-[(phenylsulphanyl)methyl]propyl}amino)-3-nitrobenzenesulphonamide by 3-nitro-4-[(3-phenylpropyl)amino]benzenesulphonamide.

### Elemental Microanalysis:

| | % C | % H | % N | % S |
|---|---|---|---|---|
| Calculated | 68.79 | 5.77 | 9.78 | 4.48 |
| Found | 68.15 | 5.85 | 9.76 | 3.63 |

### EXAMPLE 10

### 4-[(2-Anilinoethyl)amino]-N-{[(4aS,R)-3-([1,1′-biphenyl]-2-ylmethyl)-2,3,4,4a,5,6-hexahydro-1H-pyrazino[1,2-a]quinolin-8-yl]carbonyl)-3-nitrobenzenesulphonamide

The procedure is as for Step A of Example 1, replacing the compound obtained in Preparation 1 by the compound obtained in Preparation 4, and replacing 4-({(1R)-3-(dimethylamino)-1-[(phenylsulphanyl)methyl]propyl}amino)-3-nitrobenzenesulphonamide by 4-[(2-anilinoethyl)amino]-3-nitrobenzenesulphonamide.

### Elemental Microanalysis:

| | % C | % H | % N | % S |
|---|---|---|---|---|
| Calculated | 67.02 | 5.62 | 11.72 | 4.47 |
| Found | 65.93 | 5.80 | 11.61 | 3.72 |

### EXAMPLE 11

### N-{[(4aS,R)-3-[1,1′-Biphenyl]-2-ylmethyl)-2,3,4,4a,5,6-hexahydro-1H-pyrazino[1,2-a]quinolin-8-yl]carbonyl}-4-{[3-(dimethylamino)propyl]-[2-(phenylsulphanyl)ethyl]amino}-3-nitrobenzenesulphonamide hydrochloride

### Step A: N-{[(4aS,R)-3-([1,1′-Biphenyl]-2-ylmethyl)-2,3,4,4a,5,6-hexahydro-1H-pyrazino[1,2-a]quinolin-8-yl]carbonyl}-4-{[3-(dimethylamino)propyl][2-(phenylsulphanyl)ethyl]amino}-3-nitrobenzenesulphonamide

The procedure is as for Step A of Example 1, replacing the compound obtained in Preparation 1 by the compound obtained in Preparation 4, and replacing 4-({(1R)-3-(dimethylamino)-1-[(phenylsulphanyl)methyl]propyl}amino)-3-nitrobenzenesulphonamide by 4-{[3-(dimethylamino)propyl][2-(phenylsulphanyl)ethyl]amino}-3-nitrobenzene-sulphonamide.

### Step B: N-{[(4aS,R)-3-([1,1′-Biphenyl]-2-ylmethyl)-2,3,4,4a,5,6-hexahydro-1H-pyrazino[1,2-a]quinolin-8-yl]carbonyl}-4-{[3-(dimethylamino)propyl][2-(phenylsulphanyl)ethyl]amino}-3-nitrobenzenesulphonamide hydrochloride

The procedure is as for Step B of Example 2.

### Elemental Microanalysis:

| | % C | % H | % N | % S |
|---|---|---|---|---|
| Calculated | 64.55 | 6.08 | 10.04 | 7.66 |
| Found | 64.67 | 5.99 | 10.03 | 7.47 |

### EXAMPLE 12

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{[3-(dimethylamino)propyl]amino}-3-nitrobenzenesulphonamide

The procedure is as for Step A of Example 1, replacing 4-({(1R)-3-(dimethylamino)-1-[(phenylsulphanyl)methyl]propyl}amino)-3-nitrobenzenesulphonamide by 4-{[3-(dimethylamino)propyl]amino}-3-nitrobenzenesulphonamide.

### Elemental Microanalysis:

| | % C | % H | % N | % S |
|---|---|---|---|---|
| Calculated | 61.96 | 5.76 | 11.72 | 4.47 |
| Found | 62.36 | 6.08 | 11.50 | 4.94 |

### EXAMPLE 13

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)benzene-sulphonamide

The procedure is as for Step A of Example 1, replacing 4-({(1R)-3-(dimethylamino)-1-[(phenylsulphanyl)methyl]propyl}amino)-3-nitrobenzenesulphonamide by benzene-sulphonamide.

### Elemental Microanalysis:

| | % C | % H | % N | % S |
|---|---|---|---|---|
| Calculated | 67.18 | 5.29 | 7.34 | 5.60 |
| Found | 66.74 | 5.22 | 7.19 | 5.48 |

### EXAMPLE 14

### 4-{[(2-Aminoethyl)(2-phenylethyl)amino]methyl)-N-{(4aS,R)-3-[(4′-chloro-[1,1′-biphenyl]-2-yl)methyl]-2,3,4,4a,5,6-hexahydro-1H-pyrazino-[1,2-a]quinolin-8-yl)carbonyl)benzenesulphonamide tris(trifluoroacetate)

### Step A: 4-{[(2-Aminoethyl)(2-phenylethyl)amino]methyl}-N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)benzenesulphonamide

The procedure is as for Step A of Example 1, replacing 4-({(1R)-3-(dimethylamino)-1-[(phenylsulphanyl)methyl]propyl}amino)-3-nitrobenzenesulphonamide by 4-{[(2-amino-ethyl)(2-phenylethyl)amino]methyl}benzenesulphonamide.

### Step B: 4-{[(2-Aminoethyl)(2-phenylethyl)amino]methyl}-N-({(4aS,R)-3-[(4′-chloro-[1,1′-biphenyl]-2-yl)methyl]-2,3,4,4a,5,6-hexahydro-1H-pyrazino[1,2-a]quinolin-8-yl}carbonyl)benzenesulphonamide tris(trifluoroacetate)

The procedure is as for Step B of Example 1.

### Elemental Microanalysis:

| | % C | % H | % N | % S |
|---|---|---|---|---|
| Calculated | 53.97 | 4.53 | 6.42 | 2.94 |
| Found | 53.22 | 4.78 | 6.20 | 2.46 |

### EXAMPLE 15

### N-({(4aS,R)-3-[(4′-Chloro-[1,1′-biphenyl]-2-yl)methyl]-2,3,4,4a,5,6-hexahydro-1H-pyrazino[1,2-a]quinolin-7-yl}carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 7.

### Elemental Microanalysis:

| | % C | % H | % N | % S |
|---|---|---|---|---|
| Calculated | 62.95 | 5.64 | 10.01 | 7.64 |
| Found | 62.07 | 5.60 | 9.47 | 7.34 |

### EXAMPLE 16

### N-{[(4aS,R)-3-([1,1′-Biphenyl]-2-ylmethyl)-2,3,4,4a,5,6-hexahydro-1H-pyrazino[1,2-a]quinolin-7-yl]carbonyl}-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 8.

Theoretical m/z: 805.3206

Measured m/z: 805.3207

### EXAMPLE 17

### N-{[2-[1,1′-Biphenyl]-2-ylmethyl)-1,2,3,4-tetrahydropyrazino[1,2-a]indol-8-yl]carbonyl)-4-({(1R)-3-dimethylamino)-1-[(phenyl-sulphanyl)methyl]propyl}amino)-3-nitrobenzenesulphonamide bis(hydrochloride)

### Step A: N-{[2-([1,1′-Biphenyl]-2-ylmethyl)-1,2,3,4-tetrahydropyrazino[1,2-a]indol-8-yl]carbonyl}-4-({(1R)-3-(dimethylamino)-1-[(phenylsulphanyl)methyl]-propyl}amino)-3-nitrobenzenesulphonamide

The procedure is as for Step A of Example 1, replacing the compound obtained in Preparation 1 by the compound obtained in Preparation 9.

### Step B: N-{[2-([1,1′-Biphenyl]-2-ylmethyl)-1,2,3,4-tetrahydropyrazino[1,2-a]indol-8-yl]carbonyl}-4-({(1R)-3-(dimethylamino)-1-[(phenylsulphanyl)methyl]-propyl}amino)-3-nitrobenzenesulphonamide bis(hydrochloride)

The procedure is as for Step B of Example 2.

### EXAMPLE 18

### N-({2-[(4′-Chloro-[1,1′-biphenyl]-2-yl)methyl]-1,2,3,4-tetrahydro-pyrazino[1,2-a]indol-8-yl}carbonyl)4-({(1R)-3-(dimethylamino)-1-[(phenylsulphanyl)methyl]propyl}amino)-3-nitrobenzenesulphonamide bis(hydrochloride)

### Step A: N-({2-[(4′-Chloro-[1,1′-biphenyl]-2-yl)methyl]-1,2,3,4-tetrahydro-pyrazino[1,2-a]indol-8-yl}carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 10.

### Step B: N-({2-[(4′-Chloro-[1,1′-biphenyl]-2-yl)methyl]-1,2,3,4-tetrahydro-pyrazino[1,2-a]indol-8-yl}carbonyl)-4-({(1R)-3-(dimethylamino)-1-[(phenylsulphanyl)methyl]propyl}amino)-3-nitrobenzenesulphonamide bis(hydrochloride)

The procedure is as for Step B of Example 2.

### EXAMPLE 19

### N-({(10aS,R)-2-[(4′-Chloro-[1,1′-biphenyl]-2-yl)methyl]-1,2,3,4,10,10a-hexahydropyrazino[1,2-a]indol-8-yl}carbonyl)-4-({(1R)-3-dimethyl-amino)-1-[(phenylsulphanyl)methyl]propyl}amino)-3-nitrobenzene-sulphonamide

The procedure is as for Step A of Example 1, replacing the compound obtained in Preparation 1 by the compound obtained in Preparation 11.

### Elemental Microanalysis:

| | % C | % H | % N | % S |
|---|---|---|---|---|
| Calculated | 57.49 | 5.27 | 9.35 | 7.14 |
| Found | 57.31 | 5.47 | 9.06 | 6.95 |

### EXAMPLE 20

### N-({(4aR)-3-[(4′-Chloro-[1,1′-biphenyl]-2-yl)methyl]-2,3,4,4a,5,6-hexahydro-1H-pyrazino]1,2-a]quinolin-8-yl}carbonyl)-4-({(1R)-3-(dimethylamino)-1-[(phenylsulphanyl)methyl]propyl}amino)-3-nitrobenzenesulphonamide bis(hydrochloride)

### Step A: N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 13.

### Elemental Microanalysis:

| | % C | % H | % N | % S |
|---|---|---|---|---|
| Calculated | 62.95 | 5.64 | 10.01 | 7.64 |
| Found | 62.30 | 5.59 | 9.66 | 7.40 |

### Step B: N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide bis(hydrochloride)

The procedure is as for Step B of Example 2.

### Elemental Microanalysis

| | % C | % H | % N | % S |
|---|---|---|---|---|
| Calculated | 57.92 | 5.41 | 9.21 | 7.03 |
| Found | 58.44 | 5.21 | 9.19 | 6.14 |

### EXAMPLE 21

### N-({(4aS)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide bis(hydrochloride)

### Step A: N-({(4aS)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 12.

### Step B: N-({(4aS)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide bis(hydrochloride)

The procedure is as for Step B of Example 2.

### Elemental Microanalysis:

| | % C | % H | % N | % S |
|---|---|---|---|---|
| Calculated | 57.92 | 5.41 | 9.21 | 7.03 |
| Found | 57.58 | 5.29 | 8.75 | 6.97 |

### EXAMPLE 22

### N-{[(4aS)-3-([1,1′-Biphenyl]-2-ylmethyl)-2,3,4,4a,5,6-hexahydro-1H-pyrazino[1,2-a]quinolin-8-yl]carbonyl}-4-({(1R)-3-dimethylamino)-1-[(phenylsulphanyl)methyl]propyl}amino)-3-nitrobenzenesulphonamide tris(hydrochloride)

### Step A: N-{[(4aS)-3-([1,1′-Biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 14.

### Step B: N-{[(4aS)-3-([1,1′-Biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)methyl]propyl}amino)-3-nitrobenzenesulphonamide tris(hydrochloride)

The procedure is as for Step B of Example 2.

### Elemental Microanalysis:

| | % C | % H | % N | % S |
|---|---|---|---|---|
| Calculated | 59.21 | 5.69 | 9.42 | 7.19 |
| Found | 58.5 | 6.05 | 9.12 | 6.48 |

### EXAMPLE 23

### N-{[(4aR)-3-([1,1′-Biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)methyl]propyl}amino)-3-nitrobenzene-sulphonamide bis(hydrochloride)

### Step A: N-{[(4aR)-3-([1,1′-Biphenyl]-2-ylmethyl)-2,3,4,4a,5,6-hexahydro-1H-pyrazino[1,2-a]quinolin-8-yl]carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound obtained in Preparation 1 by the compound obtained in Preparation 15.

### Step B: N-{[(4aR)-3-([1,1′-Biphenyl]-2-ylmethyl)-2,3,4,4a,5,6-hexahydro-1H-pyrazino[1,2-a]quinolin-8-yl]carbonyl}-4-({(1R)-3-(dimethylamino)-1-[(phenylsulphanyl)methyl]propyl}amino)-3-nitrobenzenesulphonamide bis(hydrochloride)

The procedure is as for Step B of Example 2.

### Elemental Microanalysis

| | % C | % H | % N | % S |
|---|---|---|---|---|
| Calculated | 58.73 | 5.67 | 9.34 | 7.13 |
| Found | 58.78 | 5.9 | 9.17 | 7.29 |

### EXAMPLE 24

### N-{[(4aS,R)-3-[1,1′-Biphenyl]-2-ylsulphonyl)-2,3,4,4a,5,6-hexahydro-1H-pyrazino[1,2-a]quinolin-8-yl]carbonyl}-3-nitro-4-{[2-(phenyl-sulphanyl)ethyl]amino}benzenesulphonamide

The procedure is as for Step A of Example 1, replacing the compound of Preparation 1 by the compound of Preparation 16, and replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Elemental Microanalysis:

| | % C | % H | % N | % S |
|---|---|---|---|---|
| Calculated | 59.75 | 4.76 | 8.93 | 12.27 |
| Found | 60.02 | 4.98 | 8.41 | 11.81 |

### EXAMPLE 25

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-((1R)-3-(dimethylamino)-1-[(phenylsulphanyl)methyl]propyl}-3,4-dihydro-2H-1,2,4-benzothiadiazine-7-sulphonamide 1,1-dioxide

The procedure is as for Step A of Example 1, replacing 4-({(1R)-3-(dimethylamino)-1-[(phenylsulphanyl)methyl]propyl}amino)-3-nitrobenzenesulphonamide by 4-{(1R)-3-(dimethylamino)-1-[(phenylsulphanyl)methyl]propyl}-3,4-dihydro-2H-1,2,4-benzo-thiadiazine-7-sulphonamide 1,1-dioxide.

### EXAMPLE 26

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{(1R)-3-(dimethylamino)-1-[(phenylsulphanyl)methyl]propyl}-4H-1,2,4-benzothiadiazine-7-sulphonamide 1,1-dioxide

The procedure is as for Step A of Example 1, replacing 4-({(1R)-3-(dimethylamino)-1-[(phenylsulphanyl)methyl]propyl}amino)-3-nitrobenzenesulphonamide by 4-{(1R)-3-(dimethylamino)-1-[(phenylsulphanyl)methyl]propyl}-4H-1,2,4-benzothiadiazine-7-sulphonamide 1,1-dioxide.

### EXAMPLE 27

### N-({(4aS,R)-3-[(4′-Chloro-[1,1′-biphenyl]-2-yl)methyl]-1,2,3,4,4a,5,6,7-octahydropyrazino[1,2-a][1]benzazepin-9-yl}carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 17.

### EXAMPLE 28

### N-({(4aS)-3-[(4′-Chloro-[1,1′-biphenyl]-2-yl)methyl]-1,2,3,4,4a,5,6,7-octahydropyrazino[1,2-a][1]benzazepin-9-yl}carbonyl)-4-({(1R)-3-(dimethylamino)-1-[(phenylsulphanyl)methyl]propyl}amino)-3-nitrobenzenesulphonamide

The mixture of enantiomers described in Preparation 17 is separated over a column. The title compound is obtained by subjecting the selected enantiomer to the procedure of Step A of Example 1.

### EXAMPLE 29

### N-({(4aR)-3-[(4′-Chloro-[1,1′-biphenyl]-2-yl)methyl]-1,2,3,4,4a,5,6,7-octahydropyrazino[1,2-a][1]benzazepin-9-yl}carbonyl)-4-({(1R)-3-(dimethylamino)-1-[(phenylsulphanyl)methyl]propyl}amino)-3-nitrobenzenesulphonamide

The procedure is as for Example 28, using the other enantiomer.

### EXAMPLE 30

### N-({(4aS,R)-3-[(4′-Chloro-[1,1′-biphenyl]-2-yl)methyl]-1,2,3,4,4a,5,6,7-octahydropyrazino[1,2-a][1]benzazepin-9-yl}carbonyl)-4-({(1R)-3-(4-morpholinyl)-1-[(phenylsulphanyl)methyl]propyl}amino)-3-nitrobenzenesulphonamide

The procedure is as for Step A of Example 1, replacing the compound of Preparation 1 by the compound of Preparation 17, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-nitrobenzenesulphonamide.

### EXAMPLE 31

### N-({3-[(4′-Chloro-[1,1′-biphenyl]-2-yl)methyl]-1,2,3,4,4a,5,6,7-octahydropyrazino[1,2-a][1]benzazepin-9-yl}carbonyl)-4-({(1R)-3-(4-morpholinyl)-1-[(phenylsulphanyl)methyl]propyl}amino)-3-[(trifluoromethyl)sulphonyl]benzenesulphonamide

The procedure is as for Step A of Example 1, replacing the compound of Preparation 1 by the compound of Preparation 17, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-[(trifluoromethyl)sulphonyl]-benzenesulphonamide.

### EXAMPLE 32

### N-({(10aα)-2-[(4′-Chloro-[1,1′-biphenyl]-2-yl)methyl]-1,2,3,4,10,10a-hexahydropyrazino[1,2-a]indol-8-yl}carbonyl)-4-({(1R)-3-(dimethylamino)-1-[(phenylsulphanyl)methyl]propyl}amino)-3-nitrobenzenesulphonamide bishydrochloride (α=R or S)

The title compound is obtained according to the procedure of Step A of Example 1, replacing the compound of Preparation 1 by that of Preparation 18.

### Elemental Microanalysis:

| | % C | % H | % N | % S |
|---|---|---|---|---|
| Calculated | 57.49 | 5.27 | 9.35 | 7.14 |
| Found | 57.72 | 4.57 | 9.40 | 7.46 |

### EXAMPLE 33

### N-({(10aβ)-2-[(4′-Chloro-[1,1′-biphenyl]-2-yl)methyl]-1,2,3,4,10,10a-hexahydropyrazino[1,2-a]indol-8-yl}carbonyl)-4-({(1R)-3-(dimethylamino)-1-[(phenylsulphanyl)methyl]propyl}amino)-3-nitrobenzenesulphonamide bishydrochloride (β=S or R)

The title compound is obtained according to the procedure of Step A of Example 1, replacing the compound of Preparation 1 by that of Preparation 19.

### Elemental Microanalysis:

| | % C | % H | % N | % S |
|---|---|---|---|---|
| Calculated | 57.49 | 5.27 | 9.35 | 7.14 |
| Found | 57.31 | 4.29 | 9.48 | 7.87 |

### EXAMPLE 34

### N-({(10aα)-2-[(4′-Chloro-[1,1′-biphenyl]-2-yl)methyl]-1,2,3,4,10,10a-hexahydropyrazino[1,2-a]indol-8-yl}carbonyl)-4-({(1R)-3-(4-morpholinyl)-1-[(phenylsulphanyl)methyl]propyl}amino)-3-nitrobenzenesulphonamide bishydrochloride (α=R or S)

The procedure is as for Step A of Example 1, replacing the compound of Preparation 1 by the compound of Preparation 18 and replacing 4-({(1R)-3-(dimethylamino)-1-[(phenyl sulphanyl)methyl]propyl}amino)-3-nitrobenzenesulphonamide by 4-({(1R)-3-(4-morpholinyl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 57.47 | 5.25 | 8.94 | 6.82 |
| Found | 57.56 | 5.14 | 9.16 | 6.31 |

### EXAMPLE 35

### N-({(10aα)-2-[(4′-Chloro-[1,1′-biphenyl]-2-yl)methyl]-1,2,3,4,10,10a-hexahydropyrazino[1,2-a]indol-8-yl}carbonyl)-4-({(1R)-3-(4-morpholinyl)-1-[(phenylsulphanyl)methyl]propyl}amino)-3-[(trifluoromethyl)sulphonyl]benzenesulphonamide bishydrochloride (α=R or S)

The procedure is as for Step A of Example 1, replacing the compound of Preparation 1 by the compound of Preparation 18, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-[(trifluoromethyl)sulphonyl]-benzenesulphonamide.

### Elemental Microanalysis:

| | % C | % H | % N | % S |
|---|---|---|---|---|
| Calculated | 53.77 | 4.81 | 6.82 | 9.36 |
| Found | 53.63 | 4.82 | 7.28 | 8.79 |

### EXAMPLE 36

### N-({3-[(4′-Chloro-[1,1′-biphenyl]-3-yl)methyl]-5-oxo-2,3,4,4a,5,6-hexahydro-1H-pyrazino[1,2-a]quinoxalin-8-yl}carbonyl)-4-(}(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 21. There then follows a deprotection step wherein the residue already isolated is dissolved in a 4N HCl solution in dioxane. After neutralisation, the aqueous phase is extracted with CH₂Cl₂ and the organic phases are combined, dried over magnesium sulphate, filtered and concentrated. The resulting residue is purified by chromatography over a silica column (CH₂Cl₂/MeOH) to yield the expected product.

### EXAMPLE 37

### N-({3-[(4′-Chloro-[1,1′-biphenyl]-3-yl)methyl]-2,3,4,4a,5,6-hexahydro-1H-pyrazino[1,2-a]quinoxalin-8-yl}carbonyl)-4-({(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 20. There then follows a deprotection step wherein the residue already isolated is dissolved in a 4N HCl solution in dioxane. After neutralisation, the aqueous phase is extracted with CH₂Cl₂ and the organic phases are combined, dried over magnesium sulphate, filtered and concentrated. The resulting residue is purified by chromatography over a silica column (CH₂Cl₂/MeOH) to yield the expected product.

### EXAMPLE 38

### N-({3-[(4′-Chloro-[1,1′-biphenyl]-3-yl)methyl]-6-methyl-2,3,4,4a,5,6-hexahydro-1H-pyrazino[1,2-a]quinoxalin-8-yl}carbonyl)-4-({(1R)-3-(dimethylamino)-1-[(phenylsulphanyl)methyl]propyl}amino)-3-nitrobenzenesulphonamide

The nitrogen in the 6 position of the tricyclic moiety of the compound of Step H of Preparation 20 is deprotected using a 4N HCl solution in dioxane. After neutralisation, extraction and purification, the resulting residue is subjected to an alkylation reaction in the presence of methyl iodide and K₂CO₃. After treatment with LiOH, 3-[(4′-chloro-[1,1′-biphenyl]-2-yl)methyl]-6-methyl-2,3,4,4a,5,6-hexahydro-1H-pyrazino[1,2-a]quinoxaline-8-carboxylic acid is obtained. The latter compound is subjected to the procedure of Step A of Example 1 to obtain the title compound.

### EXAMPLE 39

### N-[((4aR)-3-{[2-(4-Chlorophenyl)-5,5-dimethyl-1-cyclohexen-1-yl]-methyl}-2,3,4,4a,5,6-hexahydro-1H-pyrazino[1,2-a]quinolin-8-yl)carbonyl]-4-({(1R)-2-(dimethylamino)-1-[(phenylsulphanyl)methyl]-ethyl}amino)-3-nitrobenzenesulphonamide bishydrochloride

The procedure is as for Step A of Example 1, replacing the compound of Preparation 1 by the compound of Preparation 23.

### Elemental Microanalysis:

| | % C | % H | % N | % S |
|---|---|---|---|---|
| Calculated | 58.50 | 6.08 | 8.90 | 6.79 |
| Found | 58.06 | 5.94 | 8.84 | 6.85 |

### EXAMPLE 40

### N-[((4aR)-3-{[2-(4-Chlorophenyl)-5,5-dimethyl-1-cyclohexen-1-yl]-methyl}-2,3,4,4a,5,6-hexahydro-1H-pyrazino[1,2-a]quinolin-8-yl)carbonyl]-4-({(1R)-3-(4-morpholinyl)-1-[(phenylsulphanyl)methyl]-propyl}amino)-3-[(trifluoromethyl)sulphonyl]benzenesulphonamide bishydrochloride

The procedure is as for Step A of Example 1, replacing the compound of Preparation 1 by the compound of Preparation 23, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-[(trifluoromethyl)sulphonyl]-benzenesulphonamide.

### Elemental Microanalysis:

| | % C | % H | % N | % S |
|---|---|---|---|---|
| Calculated | 54.82 | 5.54 | 6.52 | 8.96 |
| Found | 54.56 | 5.13 | 6.67 | 8.45 |

### EXAMPLE 41

### N-[((10aβ)-2-{[2-(4-Chlorophenyl)-5,5-dimethyl-1-cyclohexen-1-yl]methyl}-1,2,3,4,10,10a-hexahydropyrazino[1,2-a]indol-8-yl)-carbonyl]-4-({(1R)-3-(4-morpholinyl)-1-[(phenylsulphanyl)methyl]-propyl}amino)-3-[(trifluoromethyl)sulphonyl]benzenesulphonamide bishydrochloride (β=S or R)

The procedure is as for Step A of Example 1, replacing the compound of Preparation 1 by the compound of Preparation 24, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-[(trifluoromethyl)sulphonyl]-benzenesulphonamide.

### Elemental Microanalysis:

| | % C | % H | % N | % S |
|---|---|---|---|---|
| Calculated | 54.41 | 5.42 | 6.61 | 9.08 |
| Found | 53.66 | 5.51 | 6.50 | 8.71 |

### EXAMPLE 42

### N-[((4aR)-3-{[4-(4-Chlorophenyl)-3-pyridyl]methyl}-2,3,4,4a,5,6-hexahydro-1H-pyrazino[1,2-a]quinolin-8-yl)carbonyl]-4-({(1R)-2-(dimethylamino)-1-[(phenylsulphanyl)methyl]ethyl}amino)-3-nitrobenzenesulphonamide trihydrochloride

The procedure is as for Step A of Example 1, replacing the compound of Preparation 1 by the compound of Preparation 25.

### Elemental Microanalysis:

| | % C | % H | % N | % S |
|---|---|---|---|---|
| Calculated | 54.37 | 5.20 | 10.32 | 6.75 |
| Found | 54.70 | 5.12 | 10.35 | 6.71 |

### EXAMPLE 43

### N-[((4aR)-3-{[2-(4-Chlorophenyl)-3-pyridyl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 26.

### EXAMPLE 44

### N-[((4aR)-3-{[3-(4-Chlorophenyl)-2-pyridyl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 27.

### EXAMPLE 45

### N-[((4aR)-3-{[3-(4-Chlorophenyl)-4-pyridyl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide

The procedure is as for Step A of Example 1, replacing the compound of Preparation 1 by the compound of Preparation 28.

### EXAMPLE 46

### N-({(4aR)-3-[(4-Amino-4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide trihydrochloride

The procedure is as for Step A of Example 1, replacing the compound of Preparation 1 by the compound of Preparation 29. There then follows a deprotection step wherein the residue already isolated is dissolved in a 4N HCl solution in dioxane. After neutralisation, the aqueous phase is extracted with CH₂Cl₂ and the organic phases are combined, dried over magnesium sulphate, filtered and concentrated. The resulting residue is purified by chromatography over a silica column (CH₂Cl₂/MeOH) to yield the expected product.

### Elemental Microanalysis:

| | % C | % H | % N | % S |
|---|---|---|---|---|
| Calculated | 54.83 | 5.33 | 10.17 | 6.65 |
| Found | 54.97 | 5.25 | 10.07 | 6.62 |

### EXAMPLE 47

### N-[((4aR)-3-{[4-(Aminomethyl)-4′-chloro-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide trihydrochloride

The procedure is as for Step A of Example 1, replacing the compound of Preparation 1 by the compound of Preparation 30. There then follows a deprotection step wherein the residue already isolated is dissolved in a 4N HCl solution in dioxane. After neutralisation, the aqueous phase is extracted with CH₂Cl₂ and the organic phases are combined, dried over magnesium sulphate, filtered and concentrated. The resulting residue is purified by chromatography over a silica column (CH₂Cl₂/MeOH) to yield the expected product.

### Elemental Microanalysis:

| | % C | % H | % N | % S |
|---|---|---|---|---|
| Calculated | 55.27 | 5.46 | 10.03 | 6.56 |
| Found | 55.98 | 5.55 | 9.82 | 6.31 |

### EXAMPLE 48

### N-[((4aR)-3-{[3′-Fluoro-4′-chloro-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-1(phenylsulphanyl)methyl]propyl}amino)-3-nitrobenzenesulphonamide bishydrochloride

The procedure is as for Step A of Example 1, replacing the compound of Preparation 1 by the compound of Preparation 31.

### Elemental Microanalysis.

| | % C | % H | % N | % S |
|---|---|---|---|---|
| Calculated | 56.80 | 5.20 | 9.03 | 6.89 |
| Found | 56.06 | 5.20 | 9.08 | 6.55 |

### EXAMPLE 49

### N-[((4aR)-3-{[4′-Cyano-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 32.

### Elemental Microanalysis

| | % C | % H | % N | % S |
|---|---|---|---|---|
| Calculated | 59.86 | 5.47 | 10.86 | 7.10 |
| Found | 59.86 | 5.19 | 10.86 | 6.72 |

### EXAMPLE 50

### N-[((4aR)-3-{[4′-(Trifluoromethyl)-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 33.

### Elemental Microanalysis:

| | % C | % H | % N | % S |
|---|---|---|---|---|
| Calculated | 57.14 | 5.22 | 8.88 | 6.78 |
| Found | 57.72 | 5.10 | 8.83 | 6.34 |

### EXAMPLE 51

### N-[((4aS,R)-3-{[4′-(Trifluoromethyl)-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide trifluoroacetate

The procedure is as for Example 50, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1).

### EXAMPLE 52

### N-({(4aR)-3-[2-(1,3-Benzodioxol-5-yl)benzyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 34.

### Elemental Microanalysis:

| | % C | % H | % N | % S |
|---|---|---|---|---|
| Calculated | 58.62 | 5.47 | 9.12 | 6.96 |
| Found | 58.76 | 5.25 | 9.19 | 6.83 |

### EXAMPLE 53

### N-({(4aS,R)-3-[2-(1,3-Benzodioxol-5-yl)benzyl]-2,3,4,4a,5,6-hexahydro-1H-pyrazino[1,2-a]quinolin-8-yl}carbonyl-4-{[2-(phenylsulphanyl)ethyl]-amino})-3-nitrobenzenesulphonamide trifluoroacetate

The procedure is as for Example 52, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1), and using 4-{[2-(phenylsulphanyl)ethyl]amino}3-nitrobenzenesulphonamide instead of 4-({(1R)-3-(dimethylamino)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 55.64 | 4.38 | 7.4 | 6.78 |
| Found | 55.42 | 4.37 | 7.35 | 6.87 |

### EXAMPLE 54

### N-{[(4aR)-3-Benzhydryl-2,3,4,4a,5,6-hexahydro-1H-pyrazino[1,2-a]quinolin-8-yl]carbonyl}-4-({(1R)-3-(dimethylamino)-1-[(phenyl-sulphanyl)methyl]propyl}amino)-3-nitrobenzenesulphonamide

The procedure is as for Step A of Example 1, replacing the compound of Preparation 1 by the compound of Preparation 35.

### Elemental Microanalysis:

| | % C | % H | % N | % S |
|---|---|---|---|---|
| Calculated | 65.65 | 6.01 | 10.44 | 7.97 |
| Found | 64.63 | 5.91 | 10.11 | 7.81 |

### EXAMPLE 55

### N-[((4aR)-3-{2-Bromobenzyl}-2,3,4,4a,5,6-hexahydro-1H-pyrazino[1,2-a]quinolin-8-yl)carbonyl]-4-({(1R)-3-(dimethylamino)-1-[(phenyl-sulphanyl)methyl]propyl]amino)-3-nitrobenzenesulphonamide bishydrochloride

The compound obtained in Step A of Preparation 31 is subjected to the procedure of Step N of Preparation 17. The resulting product is then subjected to the procedure of Step A of Example 1.

### Elemental Microanalysis:

| | % C | % H | % N | % S |
|---|---|---|---|---|
| Calculated | 51.82 | 5.15 | 9.54 | 7.28 |
| Found | 51.33 | 5.14 | 9.66 | 7.08 |

### EXAMPLE 56

### N-[((4aS,R)-3-{2-Bromobenzyl}-2,3,4,4a,5,6-hexahydro-1H-pyrazino[1,2-a]quinolin-8-yl)carbonyl]-4-([2-(phenylsulphanyl)ethyl]amino)-3-nitrobenzenesulphonamide hydrochloride

The procedure is as for Example 55, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1), and using 4-{[2-(phenylsulphanyl)ethyl]amino}3-nitrobenzenesulphonamide instead of 4-({(1R)-3-(dimethylamino)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 52.82 | 4.56 | 9.06 | 8.29 |
| Found | 52.92 | 4.48 | 8.84 | 8.47 |

### EXAMPLE 57

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 57.89 | 5.39 | 8.81 | 6.72 |
| Found | 57.11 | 5.06 | 8.48 | 7.46 |

### EXAMPLE 58

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-nitrobenzenesulphonamide bishydrochloride

The procedure is as for Example 57, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1).

### Elemental Microanalysis:

| | % C | % H | % N | % S |
|---|---|---|---|---|
| Calculated | 57.89 | 5.39 | 8.81 | 6.72 |
| Found | 57.38 | 5.22 | 8.80 | 6.57 |

### EXAMPLE 59

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-[(trifluoromethyl)sulphonyl]benzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(4-morpholinyl)-1-[(phenylsulphanyl)methyl]propyl}amino)-3-[(trifluoromethyl)sulphonyl]-benzenesulphonamide.

### Elemental Microanalysis:

| | % C | % H | % N | % S |
|---|---|---|---|---|
| Calculated | 54.20 | 4.94 | 6.72 | 9.24 |
| Found | 54.31 | 4.58 | 6.75 | 8.90 |

### EXAMPLE 60

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1S)-3-(4-morpholinyl)-1-[(phenylsulphanyl)methyl]propyl}amino)-3-nitrobenzenesulphonamide bishydrochloride

The procedure is as for Example 59, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1), and replacing 4-({(1R)-3-(4-morpholinyl)-1-[(phenyl-sulphanyl)methyl]propyl}amino)-3-[(trifluoromethyl)sulphonyl]benzenesulphonamide by 4-({(1S)-3-(4-morpholinyl)-1-[(phenylsulphanyl)methyl]propyl}amino)-3-nitrobenzene-sulphonamide.

### Elemental Microanalysis:

| | % C | % H | % N | % S |
|---|---|---|---|---|
| Calculated | 57.89 | 5.39 | 8.81 | 6.72 |
| Found | 57.69 | 5.32 | 9.05 | 6.54 |

### EXAMPLE 61

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-methyl-1-piperazinyl)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(4-methyl-1-piperazinyl)-1-[(phenylsulphanyl)methyl]propyl}amino)-3-nitrobenzene-sulphonamide.

### EXAMPLE 62

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-piperidyl)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(1-piperidyl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 59.27 | 5.61 | 8.82 | 6.73 |
| Found | 59.23 | 5.21 | 8.75 | 6.78 |

### EXAMPLE 63

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-piperidyl)-1-[(phenylsulphanyl)methyl]propyl}amino)-3-nitrobenzenesulphonamide bishydrochloride

The procedure is as for Example 62, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1).

### Elemental Microanalysis:

| | % C | % H | % N | % S |
|---|---|---|---|---|
| Calculated | 59.27 | 5.61 | 8.82 | 6.73 |
| Found | 58.65 | 5.48 | 8.56 | 6.37 |

### EXAMPLE 64

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1S)-3-(1-piperidyl)-1-[(phenylsulphanyl)methyl]propyl}amino)-3-nitrobenzenesulphonamide bishydrochloride

The procedure is as for Example 63, replacing 4-({(1R)-3-(1-piperidyl)-1-[(phenylsulphanyl)methyl]propyl}amino)-3-nitrobenzenesulphonamide by 4-({(1S)-3-(1-piperidyl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 59.27 | 5.61 | 8.82 | 6.73 |
| Found | 59.00 | 5.39 | 8.58 | 6.70 |

### EXAMPLE 65

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-pyrrolidinyl)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(1-pyrrolidinyl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 58.88 | 5.48 | 8.96 | 6.83 |
| Found | 58.53 | 5.04 | 8.81 | 6.60 |

### EXAMPLE 66

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(3,6-dihydro-1(2H)-pyridyl)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(3,6-dihydro-1 (2H)-pyridyl)-1-[(phenylsulphanyl)methyl]propyl}amino)-3-nitrobenzene-sulphonamide.

### EXAMPLE 67

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-azepanyl)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-(1-azepanyl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 59.65 | 5.74 | 8.70 | 6.64 |
| Found | 60.01 | 5.60 | 8.40 | 6.39 |

### EXAMPLE 68

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({(1R)-3-((1R,5S)-3-azabicyclo[3.1.0]hex-3-yl)-1-[(phenylsulphanyl)methyl]-propyl}amino)-3-nitrobenzenesulphonamide bishydrochloride

The procedure is as for Step A of Example 1, replacing the compound of Preparation 1 by the compound of Preparation 13, and replacing 4-({(1R)-3-(dimethylamino)-1-[(phenylsulphanyl)methyl]propyl}amino)-3-nitrobenzenesulphonamide by 4-({(1R)-3-((1R,5S)-3-azabicyclo[3.1.0]hex-3-yl)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 59.39 | 5.41 | 8.84 | 6.75 |
| Found | 59.51 | 5.17 | 8.90 | 6.43 |

Examples 69 to 78 which follow are obtained by coupling the tricyclic compound described in Preparation 1 with the appropriate benzenesulphonamide compound according to the procedure described in Step A of Example 1:

### EXAMPLE 69

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-[(4-phenylbutyl)amino]-3-nitrobenzenesulphonamide bistrifluoroacetate

### Elemental Microanalysis:

| | % C | % H | % N | % S |
|---|---|---|---|---|
| Calculated | 58.24 | 4.73 | 7.58 | 3.47 |
| Found | 58.61 | 4.87 | 7.54 | 2.97 |

### EXAMPLE 70

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{[2-(1-methyl-1H-benzimidazol-2-yl)ethyl]amino}-3-nitrobenzenesulphonamide tristrifluoroacetate

### Elemental Microanalysis

| | % C | % H | % N | % S |
|---|---|---|---|---|
| Calculated | 50.91 | 3.83 | 8.66 | 2.83 |
| Found | 51.19 | 4.03 | 8.76 | 2.34 |

### EXAMPLE 71

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{[2-(1H-benzimidazol-2-yl)ethyl]amino}-3-nitrobenzenesulphonamide tristrifluoroacetate

### Elemental Microanalysis:

| | % C | % H | % N | % S |
|---|---|---|---|---|
| Calculated | 52.06 | 3.85 | 9.24 | 3.02 |
| Found | 52.1 | 3.9 | 9.1 | 2.38 |

### EXAMPLE 72

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({2-[(4-methoxyphenyl)sulphanyl]ethyl}amino)-3-nitrobenzenesulphonamide trifluoroacetate

### Elemental Microanalysis:

| | % C | % H | % N | % S |
|---|---|---|---|---|
| Calculated | 54.52 | 4.31 | 7.22 | 6.62 |
| Found | 53.91 | 4.51 | 7.15 | 6.32 |

### EXAMPLE 73

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{(1H-benzimidazol-2-yl)methylamino}-3-nitrobenzenesulphonamide bistrifluoroacetate

### Elemental Microanalysis:

| | % C | % H | % N | % S |
|---|---|---|---|---|
| Calculated | 52.29 | 3.77 | 9.57 | 3.13 |
| Found | 52.66 | 3.89 | 10.13 | 2.6 |

### EXAMPLE 74

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({2-[(2-methoxyphenyl)sulphanyl]ethyl}amino)-3-nitrobenzenesulphonamide hydrochloride

### Elemental Microanalysis

| | % C | % H | % N | % S |
|---|---|---|---|---|
| Calculated | 58.99 | 4.95 | 8.39 | 7.68 |
| Found | 59.14 | 4.92 | 8.45 | 7.45 |

### EXAMPLE 75

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-({2-[(2-pyridyl)sulphanyl]ethyl}amino)-3-nitrobenzenesulphonamide hydrochloride

### Elemental Microanalysis:

| | % C | % H | % N | % S |
|---|---|---|---|---|
| Calculated | 58.13 | 4.75 | 10.43 | 7.96 |
| Found | 57.62 | 4.23 | 9.96 | 7.55 |

### EXAMPLE 76

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{[2-(2-nitroanilino)ethyl]amino)-3-nitrobenzenesulphonamide hydrochloride

### Elemental Microanalysis:

| | % C | % H | % N | % S |
|---|---|---|---|---|
| Calculated | 57.69 | 4.72 | 11.77 | 3.85 |
| Found | 58.14 | 4.54 | 11.81 | 4.01 |

### EXAMPLE 77

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl)carbonyl)-4-[(2-{[2-(hydroxymethyl)phenyl]sulphanyl}ethyl)amino]-3-nitro-benzenesulphonamide hydrochloride

### Elemental Microanalysis:

| | % C | % H | % N | % S |
|---|---|---|---|---|
| Calculated | 58.99 | 4.95 | 8.39 | 7.68 |
| Found | 59.00 | 4.64 | 8.22 | 7.27 |

### EXAMPLE 78

### N-({(4aS,R)-3-[(4′-Chloro-[1,1′-biphenyl]-2-yl)methyl]-2,3,4,4a,5,6-hexahydro-1H-pyrazino[1,2-a]quinolin-8-yl}carbonyl)4-({(1S,R)-3-(dimethylamino)-1-[(phenylsulphanyl)methyl]propyl}amino)-3-nitrobenzenesulphonamide

### Elemental Microanalysis:

| | % C | % H | % N | % S |
|---|---|---|---|---|
| Calculated | 62.95 | 5.64 | 10.01 | 7.64 |
| Found | 63.37 | 5.50 | 10.00 | 7.76 |

Examples 79 to 90 which follow are obtained by coupling the tricyclic compound described in Preparation 10 with the appropriate benzenesulphonamide compound according to the procedure described in Step A of Example 1:

### EXAMPLE 79

### N-({2-[(4′-Chloro-[1,1′-biphenyl]-2-yl)methyl]-1,2,3,4-tetrahydro-pyrazino[1,2-a]indol-8-yl}carbonyl)-4-({3-(4-methyl-1-piperazinyl)-1-[(phenylsulphanyl)methyl]propyl}amino)-3-nitrobenzenesulphonamide tristrifluoroacetate

### Elemental Microanalysis:

| | % C | % H | % N | % S |
|---|---|---|---|---|
| Calculated | 50.35 | 4.12 | 7.81 | 5.11 |
| Found | 50.26 | 4.22 | 7.91 | 4.72 |

### EXAMPLE 80

### N-({2-[(4′-Chloro-[1,1′-biphenyl]-2-yl)methyl]-1,2,3,4-tetrahydro-pyrazino[1,2-a]indol-8-yl)carbonyl)-4-{[3-(dimethylamino)-1-(2-phenylethyl)propyl]amino}-3-nitrobenzenesulphonamide bishydrochloride

### Elemental Microanalysis

| | % C | % H | % N | % S |
|---|---|---|---|---|
| Calculated | 60.17 | 5.39 | 9.57 | 3.65 |
| Found | 59.55 | 5.6 | 9.64 | 2.58 |

### EXAMPLE 81

### N-({2-[(4′-Chloro-[1,1′-biphenyl]-2-yl)methyl]-1,2,3,4-tetrahydro-pyrazino[1,2-a]indol-8-yl}carbonyl)-4-({3-((3aR,6aS)-hexahydrocyclo-penta[c]pyrrol-2(1H)-yl)-1-[(phenylsulphanyl)methyl]propyl}amino)-3-nitrobenzenesulphonamide bishydrochloride

### Elemental Microanalysis:

| | % C | % H | % N | % S |
|---|---|---|---|---|
| Calculated | 59.9 | 5.34 | 8.73 | 6.66 |
| Found | 59.66 | 5.47 | 8.46 | 6.3 |

### EXAMPLE 82

### N-({2-[(4′-Chloro-[1,1′-biphenyl]-2-yl)methyl]-1,2,3,4-tetrahydro-pyrazino[1,2-a]indol-8-yl}carbonyl)-4-{[1-[(phenylsulphanyl)methyl]-3-(1-piperidyl)propyl]amino}-3-nitrobenzenesulphonamide bishydrochloride

### Elemental Microanalysis:

| | % C | % H | % N | % S |
|---|---|---|---|---|
| Calculated | 59.00 | 5.27 | 8.97 | 6.85 |
| Found | 58.95 | 5.4 | 8.68 | 6.59 |

### EXAMPLE 83

### N-({2-[(4′-Chloro-[1,1′-biphenyl]-2-yl)methyl]-1,2,3,4-tetrahydro-pyrazino[1,2-a]indol-8-yl}carbonyl)-4-{[1-[(phenylsulphanyl)methyl]-3-(1-pyrrolidinyl)propyl]amino}-3-nitrobenzenesulphonamide bishydrochloride

### Elemental Microanalysis:

| | % C | % H | % N | % S |
|---|---|---|---|---|
| Calculated | 59.06 | 5.15 | 9.18 | 7.01 |
| Found | 59.31 | 5.21 | 9.08 | 7.43 |

### EXAMPLE 84

### N-({2-[(4′-Chloro-[1,1′-biphenyl]-2-yl)methyl]-1,2,3,4-tetrahydro-pyrazino[1,2-a]indol-8-yl}carbonyl)-4-{[3-(dimethylamino)-1-(phenoxymethyl)propyl]amino}-3-nitrobenzenesulphonamide bishydrochloride

### Elemental Microanalysis:

| | % C | % H | % N | % S |
|---|---|---|---|---|
| Calculated | 58.67 | 5.15 | 9.55 | 3.64 |
| Found | 58.65 | 4.99 | 9.44 | 3.31 |

### EXAMPLE 85

### N-({2-[(4′-Chloro-[1,1′-biphenyl]-2-yl)methyl]-1,2,3,4-tetrahydro-pyrazino[1,2-a]indol-8-yl}carbonyl)-4-({3-(4-morpholinyl)-1-[(phenylsulphanyl)methyl]propyl}amino)-3-nitrobenzenesulphonamide bishydrochloride

### Elemental Microanalysis:

| | % C | % H | % N | % S |
|---|---|---|---|---|
| Calculated | 57.6 | 5.05 | 8.96 | 6.83 |
| Found | 58.45 | 5.03 | 8.78 | 6.61 |

### EXAMPLE 86

### N-({2-[(4′-Chloro-[1,1′-biphenyl]-2-yl)methyl]-1,2,3,4-tetrahydro-pyrazino[1,2-a]indol-8-yl}carbonyl)-4-{[1-(anilinomethyl)-3-(dimethyl-amino)propyl]amino}-3-nitrobenzenesulphonamide bistrifluoroacetate

It should be noted that the nitrogen of the anilinomethyl group of the benzene-sulphonamide compound is protected by a Boc function at the moment of coupling to the tricycle. The deprotection step which yields the title product is carried out in the presence of 6N HCl and dioxane.

### Elemental Microanalysis:

| | % C | % H | % N | % S |
|---|---|---|---|---|
| Calculated | 51.86 | 4.19 | 8.71 | 2.85 |
| Found | 52.17 | 4.5 | 9.55 | 2.29 |

### EXAMPLE 87

### N-({2-[(4′-Chloro-[1,1′-biphenyl]-2-yl)methyl]-1,2,3,4-tetrahydro-pyrazino[1,2-a]indol-8-yl}carbonyl)-4-({3-(dimethylamino)-1-[2-(2-furyl)ethyl]propyl}amino)-3-nitrobenzenesulphonamide bishydrochloride

### Elemental Microanalysis:

| | % C | % H | % N | % S |
|---|---|---|---|---|
| Calculated | 58.10 | 5.22 | 9.68 | 3.69 |
| Found | 57.85 | 4.90 | 9.66 | 3.70 |

### EXAMPLE 88

### N-({2-[(4′-Chloro-[1,1′-biphenyl]-2-yl)methyl]-1,2,3,4-tetrahydro-pyrazino[1,2-a]indol-8-yl}carbonyl)-4-{[1-[(phenylsulphanyl)methyl]-3-(4-thiomorpholinyl)propyl]amino}-3-nitrobenzenesulphonamide bishydrochloride

### Elemental Microanalysis:

| | % C | % H | % N | % S |
|---|---|---|---|---|
| Calculated | 56.63 | 4.96 | 8.81 | 10.08 |
| Found | 56.44 | 4.71 | 8.82 | 10.10 |

### EXAMPLE 89

### N-({2-[(4′-Chloro-[1,1′-biphenyl]-2-yl)methyl]-1,2,3,4-tetrahydro-pyrazino[1,2-a]indol-8-yl}carbonyl)-4-({3-[methoxy(methyl)amino]-1-[(phenylsulphanyl)methyl]propyl}amino)-3-nitrobenzenesulphonamide hydrochloride

### EXAMPLE 90

### N-({2-[(4′-Chloro-[1,1′-biphenyl]-2-yl)methyl]-1,2,3,4-tetrahydro-pyrazino[1,2-a]indol-8-yl}carbonyl)-4-({3-(4,4-difluoro-1-piperidyl)-1-[(phenylsulphanyl)methyl]propyl}amino)-3-nitrobenzenesulphonamide bishydrochloride

### Elemental Microanalysis:

| | % C | % H | % N | % S |
|---|---|---|---|---|
| Calculated | 56.82 | 4.87 | 8.64 | 6.60 |
| Found | 56.88 | 4.65 | 8.64 | 6.49 |

### EXAMPLE 91

### N-[((4aS,R)-3-{[4-(4-Chlorophenyl)-3-pyridyl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide hydrochloride

The procedure is as for Step A of Example 1, replacing the compound of Preparation 1 by the compound of Preparation 36 and replacing 4-({(1R)-3-(dimethylamino)-1-[(phenylsulphanyl)-methyl]propyl}amino)-3-nitrobenzene by 4-{[2-(phenylsulphanyl)ethyl]amino}-3-nitro-benzenesulphonamide.

### Elemental Microanalysis

| | % C | % H | % N | % S |
|---|---|---|---|---|
| Calculated | 58.13 | 4.75 | 10.43 | 7.96 |
| Found | 57.15 | 4.36 | 10.20 | 7.46 |

### EXAMPLE 92

### N-[((4aS,R)-3-{[2-(4-Chlorophenyl)-3-pyridyl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide bistrifluoroacetate

The procedure is as for Step A of Example 1, replacing the compound of Preparation 1 by the compound of Preparation 37 and replacing 4-({(1R)-3-(dimethylamino)-1-[(phenylsulphanyl)-methyl]propyl}amino)-3-nitrobenzene by 4-{[2-(phenylsulphanyl)ethyl]amino}-3-nitro-benzenesulphonamide.

### Elemental Microanalysis:

| | % C | % H | % N | % S |
|---|---|---|---|---|
| Calculated | 51.78 | 3.94 | 8.43 | 6.43 |
| Found | 52.38 | 4.12 | 8.47 | 6.68 |

### EXAMPLE 93

### N-[((4aS,R)-3-{[2-(4-Chlorophenyl)-3-pyridyl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide trihydrochloride

The procedure is as for Step A of Example 1, replacing the compound of Preparation 1 by the compound of Preparation 37.

### Elemental Microanalysis

| | % C | % H | % N | % S |
|---|---|---|---|---|
| Calculated | 54.37 | 5.20 | 10.32 | 6.75 |
| Found | 53.74 | 5.23 | 10.15 | 5.97 |

### EXAMPLE 94

### N-[((4aS,R)-3-{[3-(4-Chlorophenyl)-2-pyridyl]methyl}-2,3,4,4a,5,6-hexahydro-1H-pyrazino[1,2-a]quinolin-8-yl)carbonyl]-3-nitro-4-{[2-(phenylsulphanyl)ethyl]amino}benzenesulphonamide hydrochloride

### Elemental Microanalysis:

| | % C | % H | % N | % S |
|---|---|---|---|---|
| Calculated | 58.13 | 4.75 | 10.43 | 7.96 |
| Found | 57.50 | 4.46 | 10.20 | 7.94 |

The procedure is as for Step A of Example 1, replacing the compound of Preparation 1 by the compound of Preparation 38 and replacing 4-({(1R)-3-(dimethylamino)-1-[(phenylsulphanyl)-methyl]propyl}amino)-3-nitrobenzene by 4-{[2-(phenylsulphanyl)ethyl]amino}-3-nitro-benzenesulphonamide.

### EXAMPLE 95

### N-[((4aS,R)-3-{[3-(4-Chlorophenyl)-4-pyridyl]methyl}-2,3,4,4a,5,6-hexahydro-1H-pyrazino[1,2-a]quinolin-8-yl)carbonyl]-4-{[2-(phenylsulphanyl)ethyl]amino}-3-nitrobenzenesulphonamide bishydrochloride

The procedure is as for Step A of Example 1, replacing the compound of Preparation 1 by the compound of Preparation 39 and replacing 4-({(1R)-3-(dimethylamino)-1-[(phenylsulphanyl)-methyl]propyl}amino)-3-nitrobenzene by 4-{[2-(phenylsulphanyl)ethyl]amino}-3-nitro-benzenesulphonamide.

### Elemental Microanalysis:

| | % C | % H | % N | % S |
|---|---|---|---|---|
| Calculated | 55.62 | 4.67 | 9.98 | 7.71 |
| Found | 56.49 | 4.50 | 10.06 | 7.32 |

### EXAMPLE 96

### N-[((4aS,R)-3-[(2-(4-Pyridyl)-3-pyridyl)methyl]-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzene-sulphonamide trishydrochloride

The procedure is as for Step A of Example 1, replacing the compound of Preparation 1 by the compound of Preparation 40.

### Elemental Microanalysis

| | % C | % H | % N | % S |
|---|---|---|---|---|
| Calculated | 53.98 | 5.34 | 11.99 | 6.86 |
| Found | 53.34 | 5.48 | 12.68 | 5.78 |

### EXAMPLE 97

### N-[((4aS,R)-3-[(2-(6-Chloro-pyrid-3-yl)-3-pyridyl)methyl]-2,3,4,4a,5,6-hexahydro-1H-pyrazino [1,2-a]quinoline-8-carbonyl]-4-({(1R)-3-(dimethylamino)-1-[(phenylsulphanyl)methyl]propyl}amino)-3-nitrobenzenesulphonamide trishydrochloride

The procedure is as for Step A of Example 1, replacing the compound of Preparation 1 by the compound of Preparation 41.

### Elemental Microanalysis:

| | % C | % H | % N | % S |
|---|---|---|---|---|
| Calculated | 53.05 | 5.09 | 11.78 | 6.74 |
| Found | 52.69 | 5.25 | 11.59 | 5.99 |

### EXAMPLE 98

### N-[((4aS,R)-3-[(2-(6-Hydroxy-pyrid-3-yl)-3-pyridyl)methyl]-2,3,4,4a,5,6-hexahydro-1H-pyrazino[1,2-a]quinoline-8-carbonyl]-4-({(1R)-3-(dimethylamino)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 42.

### EXAMPLE 99

### N-[((4aS,R)-2-{[2-(4-Chlorophenyl)-3-pyridyl]methyl}-1,2,3,4-tetrahydropyrazino[1,2-a]indole-8-carbonyl]-4-({(1R)-3-(dimethylamino)-1-[(phenylsulphanyl)methyl]propyl}amino)-3-nitrobenzenesulphonamide bishydrochloride

The procedure is as for Step A of Example 1, replacing the compound of Preparation 1 by the compound of Preparation 43.

### Elemental Microanalysis

| | % C | % H | % N | % S |
|---|---|---|---|---|
| Calculated | 55.32 | 4.91 | 10.75 | 7.03 |
| Found | 54.79 | 4.85 | 10.47 | 6.71 |

### EXAMPLE 100

### N-[((4aS,R)-2-{[2-(6-Chloro-pyrid-3-yl)-3-pyridyl]methyl}-1,2,3,4-tetrahydropyrazino[1,2-a]indole-8-carbonyl]-4-({(1R)-3-(dimethyl-amino)-1-[(phenylsulphanyl)methyl]propyl}amino)-3-nitrobenzene-sulphonamide bishydrochloride

The procedure is as for Step A of Example 1, replacing the compound of Preparation 1 by the compound of Preparation 44.

### EXAMPLE 101

### N-[((4aS,R)-3-{[4′-Cyano-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]amino}-3-nitrobenzenesulphonamide trifluoroacetate

The procedure is as for Example 49, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1) and replacing 4-({(1R)-3-(dimethylamino)-1-[(phenylsulphanyl)-methyl]propyl}amino)-3-nitrobenzene by 4-{[2-(phenylsulphanyl)ethyl]amino}-3-nitro-benzenesulphonamide.

### EXAMPLE 102

### N-[((4aS,R)-3-{[4′-(Trifluoromethyl)-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide trifluoroacetate

The procedure is as for Example 50, replacing methyl (4aR)-2,3,4,4a,5,6-hexahydro-1H-pyrazino[1,2-a]quinoline-8-carboxylate (Step A of Preparation 23) by the mixture methyl (4aS,R)-2,3,4,4a,5,6-hexahydro-1H-pyrazino[1,2-a]quinoline-8-carboxylate (Step H of Preparation 1) and replacing 4-({(1R)-3-(dimethylamino)-1-[(phenylsulphanyl)-methyl]propyl}amino)-3-nitrobenzene by 4-{[2-(phenyl sulphanyl)ethyl]amino}-3-nitro-benzenesulphonamide.

### Elemental Microanalysis:

| | % C | % H | % N | % S |
|---|---|---|---|---|
| Calculated | 54.91 | 4.15 | 7.33 | 6.71 |
| Found | 55.12 | 4.13 | 7.02 | 6.63 |

### EXAMPLE 103

### N-[((4aS,R)-3-{[3′-(Trifluoromethyl)-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide trifluoroacetate

The procedure is as for Example 102, in the course of the synthesis replacing 4-trifluoro-methylboronic acid by 3-trifluoromethylphenylboronic acid.

### EXAMPLE 104

### N-[((4aS,R)-3-{[4′-(tert-Butyl)-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide trifluoroacetate

The compound of Preparation 45 is subjected to the procedure of Step A of Example 1, replacing 4-({(1R)-3-(dimethylamino)-1-[(phenylsulphanyl)methyl]propyl}amino)-3-nitro-benzene by 4-{[2-(phenylsulphanyl)ethyl]amino}-3-nitrobenzenesulphonamide.

### Elemental Microanalysis

| | % C | % H | % N | % S |
|---|---|---|---|---|
| Calculated | 61.12 | 5.35 | 7.75 | 7.09 |
| Found | 61.17 | 5.17 | 7.85 | 6.84 |

### EXAMPLE 105

### N-[((4aS,R)-3-{[3′,5′-Dimethyl-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide trifluoroacetate

The procedure is as for Example 104, in Preparation 45 replacing 4-tert-butylphenylboronic acid by 3,5-dimethylphenylboronic acid.

### Elemental Microanalysis:

| | % C | % H | % N | % S |
|---|---|---|---|---|
| Calculated | 57.41 | 4.89 | 7.49 | 6.86 |
| Found | 57.46 | 4.87 | 7.32 | 6.77 |

### EXAMPLE 106

### N-[((4aS,R)-3-{[2′,4′-Dimethoxy-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide trifluoroacetate

The procedure is as for Example 104, in Preparation 45 replacing 4-tert-butylphenylboronic acid by 2,4-dimethoxyphenylboronic acid.

### Elemental Microanalysis:

| | % C | % H | % N | % S |
|---|---|---|---|---|
| Calculated | 55.43 | 4.72 | 7.23 | 6.62 |
| Found | 55.5 | 4.74 | 7.13 | 6.21 |

### EXAMPLE 107

### N-[((4aS,R)-3-{[3′,4′-Dimethoxy-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide trifluoroacetate

The procedure is as for Example 104, in Preparation 45 replacing 4-tert-butylphenylboronic acid by 3,4-dimethoxyphenylboronic acid.

### Elemental Microanalysis:

| | % C | % H | % N | % S |
|---|---|---|---|---|
| Calculated | 58.20 | 4.88 | 7.71 | 7.06 |
| Found | 57.25 | 4.89 | 7.68 | 7.66 |

### EXAMPLE 108

### N-[((4aS,R)-3-{[2′,3′-Dimethoxy-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide trifluoroacetate

The procedure is as for Example 104, in Preparation 45 replacing 4-tert-butylphenylboronic acid by 2,3-dimethoxyphenylboronic acid.

### EXAMPLE 109

### N-[((4aS,R)-3-{[4′-Fluoro-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide hydrochloride

The procedure is as for Example 104, in Preparation 45 replacing 4-tert-butylphenylboronic acid by 4-fluorophenylboronic acid.

### Elemental Microanalysis:

| | % C | % H | % N | % S |
|---|---|---|---|---|
| Calculated | 60.94 | 4.99 | 8.88 | 8.13 |
| Found | 61.42 | 5.18 | 8.5 | 7.48 |

### EXAMPLE 110

### N-[((4aS,R)-3-{[3′-Fluoro-4′-chloro-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino}-3-nitrobenzenesulphonamide trifluoroacetate

The procedure is as for Example 104, in Preparation 45 replacing 4-tert-butylphenylboronic acid by 3-fluoro-4-chlorophenylboronic acid.

### EXAMPLE 111

### N-[((4aS,R)-3-{[3′,4′-Dichloro-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino)-3-nitrobenzenesulphonamide trifluoroacetate

The procedure is as for Example 104, in Preparation 45 replacing 4-tert-butylphenylboronic acid by 3,4-dichlorophenylboronic acid.

### EXAMPLE 112

### N-[((4aS,R)-3-{[4′-Methyl-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide trifluoroacetate

The procedure is as for Example 104, in Preparation 45 replacing 4-tert-butylphenylboronic acid by 4-methylphenylboronic acid.

### Elemental Microanalysis:

| | % C | % H | % N | % S |
|---|---|---|---|---|
| Calculated | 59.92 | 4.91 | 8.13 | 7.44 |
| Found | 58.77 | 4.92 | 8.07 | 6.78 |

### EXAMPLE 113

### N-[((4aS,R)-3-{[3′-Chloro-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide trifluoroacetate

The procedure is as for Example 104, in Preparation 45 replacing 4-tert-butylphenylboronic acid by 3-chlorophenylboronic acid.

### Elemental Microanalysis.:

| | % C | % H | % N | % S |
|---|---|---|---|---|
| Calculated | 57.17 | 4.45 | 7.94 | 7.27 |
| Found | 56.37 | 4.50 | 7.81 | 6.67 |

### EXAMPLE 114

### N-[((4aS,R)-3-{[3′-Fluoro-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide bistrifluoroacetate

The procedure is as for Example 104, in Preparation 45 replacing 4-tert-butylphenylboronic acid by 3-fluorophenylboronic acid.

### EXAMPLE 115

### N-[((4aS,R)-3-{[3′,4′-Difluoro-[1,1′-biphenyl]-2-yl]methyl}-2,3,4,4a,5,6-hexahydro-1H-pyrazino[1,2-a]quinolin-8-yl)carbonyl]-4-{[2-(phenylsulphanyl)ethyl]-amino}-3-nitrobenzenesulphonamide trifluoroacetate

The procedure is as for Example 104, in Preparation 45 replacing 4-tert-butylphenylboronic acid by 3,4-difluorophenylboronic acid.

### Elemental Microanalysis:

| | % C | % H | % N | % S |
|---|---|---|---|---|
| Calculated | 53 | 4.1 | 7.12 | 6.52 |
| Found | 53.21 | 4.29 | 6.97 | 6.29 |

### EXAMPLE 116

### N-[(4aS,R)-3-{[3′-Chloro-4′-fluoro-[1,1′-biphenyl]-2-yl]methyl}-2,3,4,4a,5,6-hexahydro-1H-pyrazino[1,2-a]quinolin-8-yl)carbonyl]-4-{[2-(phenyl-sulphanyl)ethyl]amino)-3-nitrobenzenesulphonamide trifluoroacetate

The procedure is as for Example 104, in Preparation 45 replacing 4-tert-butylphenylboronic acid by 3-chloro-4-fluorophenylboronic acid.

### Elemental Microanalysis:

| | % C | % H | % N | % S |
|---|---|---|---|---|
| Calculated | 53.37 | 4.00 | 7.19 | 6.58 |
| Found | 53.7 | 4.09 | 7.1 | 6.16 |

### EXAMPLE 117

### N-((4aS,R)-{3-[2-(2,2-Difluoro-1,3-benzodioxol-4-yl)benzyl]-2,3,4,4a,5,6-hexahydro-1H-pyrazino[1,2-a]quinolin-8-yl}carbonyl)-3-nitro-4-{[2-(phenylsulphanyl)-ethyl]amino}benzenesulphonamide trifluoroacetate

The procedure is as for Example 104, in Preparation 45 replacing 4-tert-butylphenylboronic acid by 2,2-difluoro-1,3-benzodioxol-4-ylboronic acid.

### Elemental Microanalysis

| | % C | % H | % N | % S |
|---|---|---|---|---|
| Calculated | 55.66 | 4.13 | 7.55 | 6.91 |
| Found | 54.95 | 3.92 | 7.51 | 7.44 |

### EXAMPLE 118

### N-((4aS,R)-{3-[2-(2,3-Dihydro-1,4-benzodioxin-6-yl)benzyl]-2,3,4,4a,5,6-hexahydro-1H-pyrazino[1,2-a]quinolin-8-yl}carbonyl)-3-nitro-4-{[2-(phenylsulphanyl)-ethyl]amino}benzenesulphonamide trifluoroacetate

The procedure is as for Example 104, in Preparation 45 replacing 4-tert-butylphenylboronic acid by 2,3-dihydro-1,4-benzodioxin-6-ylboronic acid.

### EXAMPLE 119

### N-((4aS,R)-{3-[2-(1-Naphthyl)benzyl]-2,3,4,4a,5,6-hexahydro-1H-pyrazino[1,2-a]quinolin-8-yl}carbonyl)-3-nitro-4-{[2-(phenylsulphanyl)ethyl]-amino}benzenesulphonamide trifluoroacetate

The procedure is as for Example 104, in Preparation 45 replacing 4-tert-butylphenylboronic acid by 1-naphthylboronic acid.

### Elemental Microanalysis:

| | % C | % H | % N | % S |
|---|---|---|---|---|
| Calculated | 59.07 | 4.62 | 8.41 | 6.78 |
| Found | 58.92 | 4.71 | 7.2 | 6.34 |

### EXAMPLE 120

### N-((4aS,R)-{3-[2-(2-Naphthyl)benzyl]-2,3,4,4a,5,6-hexahydro-1H-pyrazino[1,2-a]quinolin-8-yl}carbonyl)-3-nitro-4-{[2-(phenylsulphanyl)ethyl]amino}-benzenesulphonamide trifluoroacetate

The procedure is as for Example 104, in Preparation 45 replacing 4-tert-butylphenylboronic acid by 2-naphthylboronic acid.

### Elemental Microanalysis

| | % C | % H | % N | % S |
|---|---|---|---|---|
| Calculated | 59.42 | 4.65 | 7.48 | 6.85 |
| Found | 59.57 | 4.76 | 7.23 | 6.83 |

### EXAMPLE 121

### N′-((4aS,R)-{3-[(4′-Chloro-[1,1′-biphenyl]-2-yl)methyl]-2,3,4,4a,5,6-hexahydro-1H-pyrazino[1,2-a]quinolin-8-yl}carbonyl)-4-[(2-phenoxyethyl)amino]-1,3-benzene-disulphonamide trifluoroacetate

The procedure is as for Step A of Example 1, replacing 4-({(1R)-3-(dimethylamino)-1-[(phenylsulphanyl)methyl]propyl}amino)-3-nitrobenzenesulphonamide by 4-[(2-phenoxy-ethyl)-amino]-1,3-benzenedisulphonamide.

### Elemental Microanalysis:

| | % C | % H | % N | % S |
|---|---|---|---|---|
| Calculated | 53.56 | 4.33 | 7.23 | 6.62 |
| Found | 53.95 | 4.38 | 7.21 | 6.28 |

### EXAMPLE 122

### N-((4aS,R)-{3-[(4′-Chloro-[1,1′-biphenyl]-2-yl)methyl]-2,3,4,4a,5,6-hexahydro-1H-pyrazino[1,2-a]quinolin-8-yl}carbonyl)-3-nitro-4-{[3-(2-oxo-1-azepanyl)propyl]amino}benzenesulphonamide bistrifluoroacetate

The procedure is as for Step A of Example 1, replacing 4-({(1R)-3-(dimethylamino)-1-[(phenylsulphanyl)methyl]propyl}amino)-3-nitrobenzenesulphonamide by 3-nitro-4-{[3-(2-oxo-1-azepanyl)propyl]amino}benzenesulphonamide.

### EXAMPLE 123

### N-{(4aS,R)-[3-([1,1′-Biphenyl]-2-ylmethyl)-2,3,4,4a,5,6-hexahydro-1H-pyrazino[1,2-a]quinolin-8-yl]carbonyl}-6-chloro-2-[2-(phenylsulphanyl)ethyl]-3,4-dihydro-2H-1,2,4-benzothiadiazine-7-sulphonamide 1,1-dioxide trifluoroacetate

The procedure is as for Step A of Example 4, replacing 4-({(1R)-3-(dimethylamino)-1-[(phenylsulphanyl)methyl]propyl}amino)-3-nitrobenzenesulphonamide by 6-chloro-2-[2-(phenylsulphanyl)ethyl]-3,4-dihydro-2H-1,2,4-benzothiadiazine-7-sulphonamide 1,1-dioxide.

### Elemental Microanalysis:

| | % C | % H | % N | % S |
|---|---|---|---|---|
| Calculated | 53.82 | 4.26 | 7.15 | 9.82 |
| Found | 53.52 | 4.20 | 7.10 | 9.86 |

### EXAMPLE 124

### N-((4aS,R)-{3-[(4′-Chloro-[1,1′-biphenyl]-2-yl)methyl]-2,3,4,4a,5,6-hexahydro-1H-pyrazino[1,2-a]quinolin-8-yl}carbonyl)-4-(2-phenoxyethyl)-4H-1,2,4-benzothiadiazine-7-sulphonamide 1,1-dioxide trifluoroacetate

The procedure is as for Step A of Example 1, replacing 4-({(1R)-3-(dimethylamino)-1-[(phenylsulphanyl)methyl]propyl}amino)-3-nitrobenzenesulphonamide by 4-(2-phenoxy-ethyl)-4H-1,2,4-benzothiadiazine-7-sulphonamide 1,1-dioxide.

### Elemental Microanalysis:

| | % C | % H | % N | % S |
|---|---|---|---|---|
| Calculated | 55.86 | 4.23 | 7.50 | 6.87 |
| Found | 55.01 | 4.30 | 7.23 | 6.76 |

### EXAMPLE 125

### N-{(4aS,R)-[3-(2-Benzylbenzyl)-2,3,4,4a,5,6-hexahydro-1H-pyrazino[1,2-a]quinolin-8-yl]carbonyl)-4-({(1R)-3-(dimethylamino)-1-[(phenylsulphanyl)methyl]propyl}amino)-3-nitrobenzenesulphonamide hydrochloride

The procedure is as for Step A of Example 5, replacing 3-nitro-4-{[2-(phenylsulphanyl)ethyl]amino}benzenesulphonamide by 4-({(1R)-3-(dimethylamino)-1-[(phenylsulphanyl)methyl]propyl}amino)-3-nitrobenzenesulphonamide.

### Elemental Microanalysis:

| | % C | % H | % N | % S |
|---|---|---|---|---|
| Calculated | 60.59 | 5.88 | 9.42 | 7.19 |
| Found | 60.50 | 5.88 | 9.43 | 6.59 |

### EXAMPLE 126

### N-((4aS,R)-{3-[2-(4-Chlorobenzyl)benzyl]-2,3,4,4a,5,6-hexahydro-1H-pyrazino[1,2-a]quinolin-8-yl}carbonyl)-3-nitro-4-{[2-(phenylsulphanyl)ethyl]amino}-benzenesulphonamide hydrochloride

The procedure is as for Step A of Example 5, replacing the compound of Preparation 5 by the compound of Preparation 46.

### Elemental Microanalysis:

| | % C | % H | % N | % S |
|---|---|---|---|---|
| Calculated | 60.14 | 5.05 | 8.55 | 7.83 |
| Found | 59.47 | 4.85 | 8.39 | 7.55 |

### EXAMPLE 127

### N-((4aS,R)-{3-[2-Phenoxybenzyl]-2,3,4,4a,5,6-hexahydro-1H-pyrazino[1,2-a]quinolin-8-yl}carbonyl)-3-nitro-4-{[2-(phenylsulphanyl)ethyl]amino}-benzenesulphonamide hydrochloride

The procedure is as for Step A of Example 1, replacing the compound of Preparation 1 by the compound of Preparation 47, and replacing 4-({(1R)-3-(dimethylamino)-1-[(phenylsulphanyl)methyl]propyl}amino)-3-nitrobenzenesulphonamide by 3-nitro-4-{[2-(phenylsulphanyl)ethyl]amino}benzenesulphonamide.

### Elemental Microanalysis:

| | % C | % H | % N | % S |
|---|---|---|---|---|
| Calculated | 61.10 | 5.13 | 8.91 | 8.16 |
| Found | 61.46 | 4.96 | 8.92 | 7.83 |

### EXAMPLE 128

### N-((4aS,R)-{3-[2-Phenoxybenzyl]-2,3,4,4a,5,6-hexahydro-1H-pyrazino[1,2-a]quinolin-8-yl}carbonyl)-3-nitro-4-({(1R)-3-(dimethylamino)-1-[(phenylsulphanyl)methyl]propyl}amino) bishydrochloride

The procedure is as for Step A of Example 1, replacing the compound of Preparation 1 by the compound of Preparation 47.

### Elemental Microanalysis

| | % C | % H | % N | % S |
|---|---|---|---|---|
| Calculated | 59.12 | 5.64 | 9.40 | 7.17 |
| Found | 59.30 | 5.34 | 9.34 | 7.23 |

### EXAMPLE 129

### N-({(4aR)-3-[(4′-Chloro-[1,1′-biphenyl]-2-yl)methyl]-2,3,4,4a,5,6-hexahydro-1H-pyrazino[1,2-a]quinolin-8-yl}carbonyl)-4-{[(3R)-3-amino-4-(phenylsulphanyl)butyl]amino}-3-nitrobenzenesulphonamide tristrifluoroacetate

The procedure is as for Example 20, replacing 4-({(1R)-3-(dimethylamino)-1-[(phenylsulphanyl)methyl]propyl}amino)-3-nitrobenzenesulphonamide by 4-{[(3R)-3-amino-4-(phenylsulphanyl)butyl]amino}-3-nitrobenzenesulphonamide.

### Elemental Microanalysis:

| | % C | % H | % N | % S |
|---|---|---|---|---|
| Calculated | 49.98 | 4.02 | 7.29 | 5.56 |
| Found | 48.89 | 3.99 | 6.92 | 5.24 |

### EXAMPLE 130

### Sodium N-{(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide

This compound is obtained starting from a solution of the bis(hydrochloride) salt described in Example 20 and subjecting it to three equivalents of NaOH.

### Pharmacological Study

### EXAMPLE A

### Induction of Caspase Activity In Vitro

This study was carried out on three human tumour cell lines:
1 small-cell lung carcinoma, H146,
1 acute myeloid leukaemia, MV4; 11,
1 leukaemia, RS4; 11.

The cell lines are cultured in an incubator at 37° C. in the presence of 5% CO₂. The H146 and RS4;11 cells are cultured in complete RPMI 1640 medium containing 10% foetal calf serum, 2 mM of glutamine, 50 units/ml of penicillin, 50 μg/ml of streptomycin and 10 mM of Hepes buffer, pH=7.4.

The MV4;11 cells are cultured in a similar medium supplemented with GM-CSF at 5 ng/ml.

The cells are distributed onto 6-well plates and exposed to the test compounds for 6 hours. They are then harvested and lysed and the caspase activity is measured in the cell lysates. This enzymatic measurement is carried out by measuring the appearance of a fluorigenic cleavage product (Pharmacia).

The results show that the compounds of the invention are powerful apoptosis inducers, evaluated by measuring caspase 3 activity in the three tumour lines tested.

By way of example, the compound of Example 20 shows an activity at 3 μM of 22000 IU in the H146 cells, an activity at 0.1 μM of 20000 IU in the MV4;11 cells and an activity at 1 μM of 23000 IU in the RS4;11 cells.

### EXAMPLE B

### Cytotoxicity In Vitro

The cytotoxicity studies were carried out on the three tumour lines of Example A. The cells are distributed onto microplates and exposed to the test compounds for 48 hours. The cell viability is then quantified by a colorimetric assay, the Microculture Tetrazolium Assay (Cancer Res., 1987, 47, 939-942).

The results are expressed in IC₅₀ (concentration of compound that inhibits cell viability by 50%), and show that the compounds of the invention are cytotoxic.

By way of Example, the compound of Example 20 has an IC₅₀ of 3.05×10⁻⁷M on H146, 2.95×10⁻⁸M on MV4;11 and 1.65×10⁻⁸M on RS4;11.

### EXAMPLE C

### Induction of Caspase Activity In Vivo

The capacity of the compounds of the invention to activate caspase 3 is evaluated on a xenotransplant model of H146 small-cell lung carcinoma cells.

5×10⁶ H146 cells are transplanted sub-cutaneously into immunosuppressed mice (NOD SCID strain). 25 to 30 days after the transplant, the test compounds are injected by the intra-peritoneal route in a mixture of Tween 80/water. Sixteen hours after treatment, the tumour masses are recovered and lysed and the caspase 3 activity is measured in the tumour lysates.

The results obtained show that the compounds of the invention are capable of inducing apoptosis in H146 tumour lines in vivo.

By way of Example, activation of more than 700% compared with the control is obtained for the compound of Example 20 and the compound of Example 23.

### EXAMPLE D

### Anti-Tumour Activity In Vivo

The anti-tumour activity of the compounds of the invention is evaluated in a xenotransplant model of H146 small-cell lung carcinoma cells.

5×10⁶ H146 cells are transplanted sub-cutaneously into immunosuppressed mice (NOD SCID strain). 25 to 30 days after the transplant, when the tumour mass has reached approximately 150 mm³, the test compounds are injected intra-peritoneally (in a mixture of Tween 80/water) every day for 21 days. The tumour mass is measured twice a week from the commencement of treatment.

The results obtained demonstrate that the compounds of the invention are capable of inducing tumour regression during the period of treatment.

By way of Example, the compound of Example 20 administered in a dose of 100 mg/kg induces almost complete tumour regression during the period of treatment, the effect persisting at least 40 days after the end of treatment.

### EXAMPLE E

### Platelet Toxicity

BDF1 mouse blood is drawn in a citrated tube, diluted in PBS and incubated in the presence of different concentrations of the test products. After 4 hours' incubation at 37° C., 20 μl of fluorescent balls (1036 balls/μl) are added to each sample. Morphological analysis by flow cytometry makes it possible to identify the platelets and counting 200 fluorescent balls makes it possible to quantify the absolute number of platelets per μl of blood analysed. In parallel, labelling with annexin V FITC followed by analysis by cytometry makes it possible to determine the percentage of platelets in apoptosis.

The compounds of the invention demonstrate acceptable platelet toxicity for development for the indication of cancer.

### EXAMPLE F

### Pharmaceutical Composition: Tablets

| | |
|---|---|
| 1000 tablets containing a dose of 5 mg of | 5 g |
| N-({(4aR)-3-[(4′-chloro-[1,1′-biphenyl]-2-yl)methyl]-2,3,4,4a,5,6- | |
| hexahydro-1H-pyrazino[1,2-a]quinolin-8-yl}carbonyl)-4-({(1R)-3- | |
| (dimethyl-amino)-1-[(phenylsulphanyl)methyl]propyl}amino)-3- | |
| nitrobenzene-sulphonamide bis(hydrochloride) (Example 20) | |
| Wheat starch | 20 g |
| Maize starch | 20 g |
| Lactose | 30 g |
| Magnesium stearate | 2 g |
| Silica | 1 g |
| Hydroxypropylcellulose | 2 g |

1. A compound selected from those of formula (I): 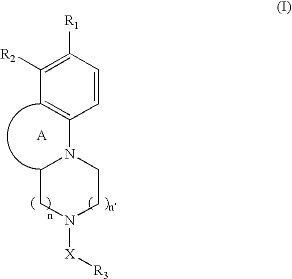
wherein
A represents a 5, 6 or 7-membered aromatic or non-aromatic ring having 1 or 2 hetero atoms selected from oxygen, sulphur and nitrogen, wherein the nitrogen atom may optionally be substituted by a linear or branched (C₁-C₆)alkyl group, it being understood that ring A cannot contain 2 sulphur atoms or 2 oxygen atoms and that one of the ring members may be a C═O group,
n and n′, which may be identical or different, represent 0, 1 or 2, where 0<n+n′<4,
R₃ represents an aryl or heteroaryl group,
X represents a linear or branched alkylene chain having from 1 to 6 carbon atoms, wherein one or two of which carbon atoms may be replaced by an oxygen atom, a cycloalkylene group, an arylene group, a heteroarylene group or an SO₂ group,
one of R₁ and R₂ represents a hydrogen atom and the other represents a group of formula (II): 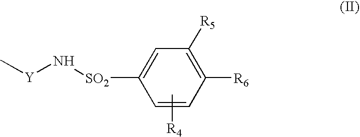
wherein:
Y represents a C═O or CH₂ group,
R₅ represents a hydrogen atom and R6 represents a hydrogen atom or an —NR₇R′₇ or —CH₂—NR₇R′₇ group wherein each of R₇ and R′₇, which may be identical or different, independently represents a hydrogen atom or a linear or branched (C₁-C₆)alkyl group substituted by one or more aryl, heteroaryl, aryloxy, heteroaryloxy, arylthio, heteroarylthio, heterocycloalkyl or —NR₁₀R′₁₀ groups wherein:
R₁₀ and R′₁₀, which may be identical or different, are selected from hydrogen, linear or branched (C₁-C₆)alkyl, linear or branched (C₁-C₆)alkoxy, aryl and heteroaryl, or
R₁₀ and R′₁₀ form a saturated or unsaturated cyclic or bicyclic group which may contain a hetero atom selected from oxygen, nitrogen and sulphur, it being understood that one or more of the ring members may represent a C═O group, and wherein the cyclic or bicyclic group may be optionally substituted by from 1 to 3 groups selected from linear or branched (C₁-C₆)alkyl optionally substituted by a hydroxy or amino group, linear or branched (C₁-C₆)alkoxy, hydroxy, carboxy, formyl, nitro, cyano, amino, linear or branched polyhalo-(C₁-C₆)alkyl, alkoxycarbonyl and halogen atoms,
or R₅ and R₆, together with the two carbon atoms carrying them, form an aromatic or non-aromatic ring having 5 or 6 ring members, one nitrogen atom of which being in the position para to the SO₂ group, and which may contain in addition to the nitrogen atom a further nitrogen atom and/or an SO₂ group, wherein the ring may be substituted by an R₇ group as defined above,
R₄ represents a halogen atom or an NO₂, R₈, SO₂—R₉, linear or branched (C₁-C₆)-alkyl or linear or branched (C₁-C₆)alkoxy group, wherein R₈ a hydrogen atom or a linear or branched (C₁-C₆)alkyl group substituted by one or more aryl, heteroaryl, aryloxy, heteroaryloxy, arylthio, heteroarylthio, heterocycloalkyl or —NR₁₀R′₁₀,
R₉ represents an amino group or a linear or branched (C₁-C₆)alkyl group optionally substituted by one or more halogen atoms,
it being understood that:
“aryl” means a phenyl, naphthyl or biphenyl group,
“heteroaryl” means a mono- or bi-cyclic group having at least one aromatic moiety and having from 5 to 10 ring members which may contain from 1 to 3 hetero atoms selected from oxygen, sulphur and nitrogen,
“heterocycloalkyl” means a mono- or bi-cyclic non-aromatic group having from 4 to 10 ring members which may contain from 1 to 3 hetero atoms selected from oxygen, sulphur and nitrogen,
“cycloalkyl” means a mono- or bi-cyclic non-aromatic group containing from 4 to 10 ring members,
wherein the aryl, heteroaryl, heterocycloalkyl and cycloalkyl may be optionally substituted by from 1 to 3 groups selected from linear or branched (C₁-C₆)alkyl optionally substituted by a hydroxy or amino group, linear or branched (C₁-C₆)alkoxy, hydroxy, carboxy, formyl, nitro, cyano, amino, linear or branched polyhalo-(C₁-C₆)alkyl, alkoxycarbonyl and halogen atoms, and
“arylene”, “heteroarylene” and “cycloalkylene” mean, respectively, a bivalent aryl, heteroaryl or cycloalkyl group as defined above,
its enantiomers and diastereoisomers, and addition salts thereof with a pharmaceutically acceptable acid or base.

2. The compound of claim 1, wherein Y represents a C═O group.

3. The compound of claim 1, wherein n and n′ represent 1.

4. The compound of claim 1, wherein R₄ represents a NO₂ or SO₂—CF₃ group.

5. The compound of claim 1, wherein X—R₃ represents a ([1,1′-biphenyl]-2-yl)methyl group optionally substituted by one or more groups selected from halogen, cyano, amino, aminomethyl and trifluoromethyl.

6. The compound of claim 1, wherein R₅ represents a hydrogen atom.

7. The compound of claim 1, wherein R₇ represents 1-(N,N-dimethylamino)-4-(phenylsulphanyl)-butan-3-yl.

8. The compound of claim 1, wherein R₇ represents a 1-(NR₁₀R′₁₀)-4-(phenylsulphanyl)-butan-3-yl group, R₁₀ and R′₁₀ being such that they form a saturated or unsaturated cyclic or bicyclic group, optionally containing a hetero atom selected from oxygen, nitrogen and sulphur.

9. The compound of claim 1, wherein R′₇ represents a hydrogen atom.

10. The compound of claim 1 which is selected from:
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenyl-sulphanyl)methyl]propyl}amino)-3-nitrobenzenesulphonamide,
N-({(10aα)-2-[(4′-chloro-[1,1′-biphenyl]-2-yl)methyl]-1,2,3,4,10,10a-hexahydro-pyrazino[1,2-a]indol-8-yl}carbonyl)-4-({(1R)-3-(dimethylamino)-1-[(phenylsulphanyl)methyl]propyl}amino)-3-nitrobenzenesulphonamide,
N-({(10aβ)-2-[(4′-chloro-[1,1′-biphenyl]-2-yl)methyl]-1,2,3,4,10,10a-hexahydro-pyrazino[1,2-a]indol-8-yl}carbonyl)-4-({(1R)-3-(dimethylamino)-1-[(phenyl-sulphanyl)methyl]propyl}amino)-3-nitrobenzenesulphonamide,
N-({(10aα)-2-[(4′-chloro-[1,1′-biphenyl]-2-yl)methyl]-1,2,3,4,10,10a-hexahydro-pyrazino[1,2-a]indol-8-yl}carbonyl)-4-({(1R)-3-(4-morpholinyl)-1-[(phenyl-sulphanyl)methyl]propyl}amino)-3-nitrobenzenesulphonamide,
N-({(10aα)-2-[(4′-chloro-[1,1′-biphenyl]-2-yl)methyl]-1,2,3,4,10,10a-hexa-hydropyrazino[1,2-a]indol-8-yl}carbonyl)-4-({(1R)-3-(4-morpholinyl)-1-[(phenylsulphanyl)methyl]propyl}amino)-3-[(trifluoromethyl)sulphonyl]benzene-sulphonamide,
N-[((4aR)-3-{[2-(4-chlorophenyl)-5,5-dimethyl-1-cyclohexen-1-yl]methyl}-2,3,4,4a,5,6-hexahydro-1H-pyrazino[1,2-a]quinolin-8-yl)carbonyl]-4-({(1R)-3-(4-morpholinyl)-1-[(phenylsulphanyl)methyl]propyl}amino)-3-[(trifluoromethyl)-sulphonyl]benzenesulphonamide,
N-[((10aβ)-2-{[2-(4-chlorophenyl)-5,5-dimethyl-1-cyclohexen-1-yl]methyl}-1,2,3,4,10,10a-hexahydropyrazino[1,2-a]indol-8-yl)carbonyl]-4-({(1R)-3-(4-morpholinyl)-1-[(phenylsulphanyl)methyl]propyl}amino)-3-[(trifluoromethyl)-sulphonyl]benzenesulphonamide,
N-[((4aR)-3-{[4-(4-chlorophenyl)-3-pyridyl]methyl}-2,3,4,4a,5,6-hexahydro-1H-pyrazino[1,2-a]quinolin-8-yl)carbonyl]-4-({(1R)-2-(dimethylamino)-1-[(phenylsulphanyl)methyl]ethyl}amino)-3-nitrobenzenesulphonamide,
N-({(4aR)-3-[(4-amino-4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzene-sulphonamide,
N-[((4aR)-3-{[4-(aminomethyl)-4′-chloro-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzene-sulphonamide trihydrochloride,
N-[((4aR)-3-{[3′-fluoro-4′-chloro-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzene-sulphonamide,
N-[((4aR)-3-{[4′-(trifluoromethyl)-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide,
N-[((4aR)-3-{[4′-cyano-[1,1′-biphenyl]-2-yl]methyl}-2,3,4,4a,5,6-hexahydro-1H-pyrazino[1,2-a]quinolin-8-yl)carbonyl]-4-({(1R)-3-(dimethylamino)-1-[(phenylsulphanyl)methyl]propyl}amino)-3-nitrobenzenesulphonamide,
N-({(4aR)-3-[2-(1,3-benzodioxol-5-yl)benzyl]-2,3,4,4a,5,6-hexahydro-1H-pyrazino[1,2-a]quinolin-8-yl}carbonyl)-4-({(1R)-3-(dimethylamino)-1-[(phenylsulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-morpholinyl)-1-[(phenylsulphanyl)methyl]propyl}amino)-3-[(trifluoromethyl)sulphonyl]-benzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(4-methyl-1-piperazinyl)-1-[(phenylsulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-piperidyl)-1-[(phenylsulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-pyrrolidinyl)-1-[(phenylsulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(3,6-dihydro-1(2H)-pyridyl)-1-[(phenylsulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-(1-azepanyl)-1-[(phenyl-sulphanyl)methyl]propyl}amino)-3-nitrobenzenesulphonamide,
N-({(4aR)-3-[(4′-chloro-[1,1′-biphenyl]-2-yl)methyl]-2,3,4,4a,5,6-hexahydro-1H-pyrazino[1,2-a]quinolin-8-yl}carbonyl)-4-({(1R)-3-((1R,5S)-3-azabicyclo-[3.1.0]hex-3-yl)-1-[(phenylsulphanyl)methyl]propyl}amino)-3-nitrobenzene-sulphonamide,
and addition salts thereof with a pharmaceutically acceptable acid or base.

11. The compound of claim 1 which is selected from N-({(4aR)-3-[(4′-chloro-[1,1′-biphenyl]-2-yl)methyl]-2,3,4,4a,5,6-hexahydro-1H-pyrazino[1,2-a]quinolin-8-yl}-carbonyl)-4-({(1R)-3-(dimethylamino)-1-[(phenylsulphanyl)methyl]propyl}amino)-3-nitrobenzenesulphonamide, and addition salts thereof with a pharmaceutically acceptable acid or base.

12. The compound of claim 1 which is N-({(4aR)-3-[(4′-chloro-[1,1′-biphenyl]-2-yl)methyl]-2,3,4,4a,5,6-hexahydro-1H-pyrazino[1,2-a]quinolin-8-yl}-carbonyl)-4-({(1R)-3-(dimethylamino)-1-[(phenylsulphanyl)methyl]propyl}amino)-3-nitrobenzenesulphonamide bis(hydrochloride).

13. The compound of claim 1 which is sodium N-({(4aR)-3-[(4′-chloro-[1,1′-biphenyl]-2-yl)methyl]-2,3,4,4a,5,6-hexahydro-1H-pyrazino[1,2-a]quinolin-8-yl}-carbonyl)-4-({(1R)-3-(dimethylamino)-1-[(phenylsulphanyl)methyl]propyl}amino)-3-nitrobenzenesulphonamide.

14. A pharmaceutical composition comprising as active ingredient a compound of claim 1, or an addition salt thereof with a pharmaceutically acceptable acid or base, in combination with one or more pharmaceutically acceptable excipients.

15. A method of treating a living animal body, including a human, afflicted with a condition involving a defect in apoptosis, comprising the step of administering to the living animal body, including a human, an amount of a compound of claim 1 which is effective for treatment of the condition.

16. A method of treating a living animal body, including a human, afflicted with cancer, comprising the step of administering to the living animal body, an amount of a compound of claim 1 which is effective for treatment of cancer.

17. The method of claim 16, wherein the cancer is selected from cancers of the bladder, brain, breast and uterus, chronic lymphoid leukaemias, cancers of the colon, oesophagus and liver, lymphoblastic leukaemias, follicular lymphomas, melanomas, malignant haemopathies, myelomas, ovarian cancers, non-small-cell lung cancers, prostate cancers and small-cell lung cancers.

18. A composition comprising a combination of a compound of claim 1 and an anti-cancer agent selected from genotoxic agents, mitotic poisons, anti-metabolites, proteasome inhibitors and kinase inhibitors.

19. A method of treating a living animal body, including a human, afflicted with cancer, comprising the step of administering to the living animal body, including a human, an amount of a composition of claim 18 which is effective for treatment of cancer.

20. A method of treating a living animal body, including a human, afflicted with cancer, comprising the step of adminstering to the living animal body, including a human, an amount of a compound of claim 1 which is effective for treatment of cancer in combination with radiotherapy.
